(19) United States
(12) Patent Application Publication
(10) Pub. No.: US 2024/0130976 A1
(43) Pub. Date: Apr. 25, 2024

(54) Pharmaceutical Formulation of Odevixibat

(71) Applicant: Albireo AB, Göteborg (SE)

(72) Inventors: Eva Byröd, Mölndal (SE); Per-Göran Gillberg, Mölndal (SE); Anna-Maria Tivert, Göteborg (SE); Rikard Bryland, Limhamn (SE); Ann-Charlotte Dahlquist, Lund (SE); Jessica Elversson, Dalby (SE); Nils Ove Gustafsson, Löddeköpinge (SE); Robert Lundqvist, Hälsö (SE); Ingvar Ymen, Saltsjö-Boo (SE); Martin Bohlin, Johanneshov (SE)

(21) Appl. No.: 18/476,590

(22) Filed: Sep. 28, 2023

Related U.S. Application Data
(63) Continuation of application No. 17/113,966, filed on Dec. 7, 2020, now Pat. No. 11,801,226.
Application No. 17/113,966 is a continuation of application No. 16/477,160, filed on Jul. 10, 2019, now Pat. No. 11,802,115.

(30) Foreign Application Priority Data
Jun. 20, 2018 (SE) 1850761-6
Jun. 20, 2018 (SE) 1850762-4

(51) Int. Cl. A61K 9/50; A61K 9/00; A61K 9/48; A61K 31/554

(52) U.S. Cl. CPC A61K 9/5047; A61K 9/009; A61K 9/4816; A61K 9/5089; A61K 31/554

(57) ABSTRACT

The invention relates to a pharmaceutical formulation, e.g. a paediatric formulation, of odevixibat, which comprises a plurality of small particles. The formulation may be used in the treatment of liver diseases such as bile acid-dependent liver diseases, and particularly cholestatic liver diseases such as biliary atresia, progressive familial intrahepatic cholestasis (PFIC), Alagille syndrome (ALGS) and paediatric cholestatic pruritus. The invention also relates to a process for the preparation of the pharmaceutical formulation.

### CROSS-REFERENCE TO RELATED APPLICATIONS

This application is a continuation of U.S. application Ser. No. 17/113,966, filed Dec. 7, 2020, which is a continuation of U.S. application Ser. No. 16/477,160, filed Jul. 10, 2019, which is a National Stage Application under 35 U.S.C. § 371 of International Application No. PCT/SE2019/050603, filed Jun. 20, 2019, which claims priority to Swedish Application No. 1850761-6, filed Jun. 20, 2018, and to Swedish Application No. 1850762-4, filed Jun. 20, 2018, the disclosures of which are incorporated by reference herein in their entireties.

### SEQUENCE LISTING

This application contains a Sequence Listing that has been submitted electronically as an XML file named “23854-0053004_SL_ST26.XM L.” The XML file, created on Sep. 27, 2023, is 16,384 bytes in size. The material in the XML file is hereby incorporated by reference in its entirety.

### TECHNICAL FIELD

The invention relates to a pharmaceutical formulation, e.g. a paediatric formulation, of odevixibat, which comprises a plurality of small particles. The formulation may be used in the treatment of liver diseases, such as bile acid-dependent liver diseases, and particularly cholestatic liver diseases such as biliary atresia, progressive familial intrahepatic cholestasis (PFIC), Alagille syndrome (ALGS) and paediatric cholestatic pruritus. The invention also relates to a process for the preparation of the pharmaceutical formulation.

BACKGROUND

The compound 1,1-dioxo-3,3-dibutyl-5-phenyl-7-methylthio-8-(N-{(R)-α-[N-((S)-1-carboxypropyl) carbamoyl]-4-hydroxybenzyl}carbamoyl methoxy)-2,3,4,5-tetrahydro-1,2,5-benzothiadiazepine (odevixibat; also known as A4250) is disclosed in WO 03/022286. The structure of odevixibat is shown below.

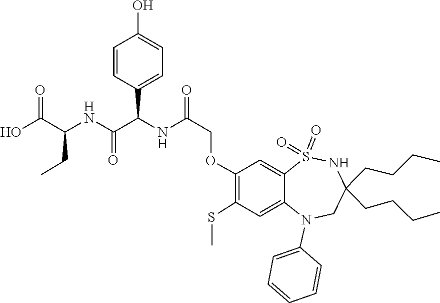 [SMILES: CCCCC1(CCCC)CN(c2ccccc2)c2cc(SC)c(OCC(=O)N[C@@H](C(=O)N[C@@H](CC)C(=O)O)c3ccc(O)cc3)cc2S(=O)(=O)N1]

As an inhibitor of the ileal bile acid transport (IBAT) mechanism, odevixibat inhibits the natural reabsorption of bile acids from the ileum into the hepatic portal circulation. Bile acids that are not reabsorbed from the ileum are instead excreted into the faeces. The overall removal of bile acids from the enterohepatic circulation leads to a decrease in the level of bile acids in serum and the liver. Odevixibat, or a pharmaceutically acceptable salt thereof, is therefore useful in the treatment of liver diseases that are associated with elevated bile acid levels, and particularly in the treatment of rare paediatric cholestatic liver diseases.

Odevixibat exhibits high potency and should be administered in low doses, such as ranging from about 40 to about 120 μg/kg. This corresponds to doses as low as 200 to 800 μg in the treatment of paediatric patients that weigh about 5 to 20 kg (e.g., infants and toddlers). It is desirable that a formulation of odevixibat can be administered to young patients in a dosage form having a small size. It is further desirable that such a formulation has good palatability, is not perceived as gritty, and is well-tolerated by infants and small children.

Multiparticulates can be administered to infants from birth if they are administered with a liquid. For children aged approximately 6 months and older (i.e. after weaning), the multiparticulates can be administered in their solid form either directly into the mouth or mixed with semi-solid food. Particle size, shape, texture, hardness, taste and dose volume (i.e., the number of particles) have been reported to be important for acceptability of multiparticulates by infants and children (Kozarewicz, Int. J. Pharm. 2014, vol. 469, pp 245-248). Various literature reviews have been conducted on the acceptability of different oral dosage forms in paediatric and older adult patients (see e.g. Liu, et al., Drugs 2014, vol. 74, pp. 1871-1889; Drumond et al., Int. J. Pharm. 2017, vol. 521, pp. 294-305; Mistry et al., J. Pharm. Pharmacol. 2017, vol. 69, pp. 361-376; Walsh et al., Int. J. Pharm. 2017, vol. 536, pp. 547-562), but the size and/or dose volume (amount) of multiparticulates investigated have not always been reported in these reviews.

Perception of grittiness may be influenced by a range of factors including particle size, quantity and dosing vehicle (see Mishra et al., Yakugaku Zasshi 2009, vol. 129, pp. 1537-1544; Lopez et al., Eur. J. Pharm. Sci. 2016, vol. 92, pp. 156-162) as well as the hardness and shape of the particles (Tyle, Acta Psychologica 1993, vol. 84, pp. 111-118), with irregular particles being perceived as larger than round (spherical) particles of the same size (Engelen et al., J. Text. Studies 2005, vol. 36, pp. 373-386). Grittiness perception studies have shown that grittiness scores may increase with increasing size and dose of the multiparticulates, whereas grittiness scores may decrease with increasing vehicle viscosity (Lopez et al., Eur. J. Pharm. Sci. 2016, vol. 92, pp. 156-162).

Capsules can be acceptable for children from approximately 6 years of age. The swallowability of the capsules can depend upon the dosage form dimensions (i.e. the size) and the ability of the child. The size, shape, taste and after taste are important capsule attributes that can influence patient acceptability (Kozarewicz, Int. J. Pharm. 2014, vol. 469, pp 245-248). In some embodiments, the size of the capsules is kept as small as possible, and the number of capsules required per dose is kept to a minimum, e.g. not more than 1-3 capsules.

In view of the above, there is a need for a formulation of odevixibat that can be easily administered in small doses adapted to the patients' weight. In some embodiments, the formulation should be suitable for treating very young patients, should be easy to swallow, and should not be perceived as gritty.

### BRIEF DESCRIPTION OF THE DRAWINGS

FIG. 1 shows the X-ray powder diffractogram of dried crystal modification 1.

FIG. 2 shows the X-ray powder diffractogram of an overhydrated sample of crystal modification 1.

FIG. 3 shows the drying of crystal modification 1, with the X-ray powder diffractogram of an overhydrated sample of crystal modification 1 at the bottom and of a dried sample at the top (2θ range 5-13°).

FIG. 4 shows the drying of crystal modification 1, with the X-ray powder diffractogram of an overhydrated sample of crystal modification 1 at the bottom and of a dry sample at the top (2θ range 18-25°).

FIG. 5 shows the X-ray powder diffractogram of crystal modification 2A, as obtained from a mixture of ethanol and water (70:30%v/v).

FIG. 6 shows the DSC trace of a sample of odevixibat with about 50% crystalline fraction (after pre-heating and cooling).

### DETAILED DESCRIPTION OF THE INVENTION

Provided herein is a multiparticulate formulation containing low doses of odevixibat. In some embodiments, the formulation is a paediatric formulation. In some embodiments, the formulation enables weight-based dosing and can be sprinkled onto food. The formulation can be designed to have a good palatability, with an optimal balance between particle size and dose volume.

In a first aspect, the invention relates to a pharmaceutical formulation of odevixibat, comprising a plurality of particles, wherein each particle contains odevixibat, or a pharmaceutically acceptable salt thereof, in an amount of from about 0.1% w/w to about 5.0% w/w based on the total weight of the particle.

Because of the low doses in which odevixibat is to be administered, and further because of the multiparticulate form of the application, each particle of the formulation contains only a very low amount of the active ingredient. For example, the amount of odevixibat, or a pharmaceutically acceptable salt thereof, in each particle can be from about 0.2% w/w to about 3.5% w/w, preferably from about 0.3% w/w to about 3.0% w/w, more preferably from about 0.4% w/w to about 2.5% w/w, and most preferably from about 0.5% w/w to about 2.0% w/w based on the total weight of the particle. In one preferred embodiment, each particle contains odevixibat, or a pharmaceutically acceptable salt thereof, in an amount of about 0.5% w/w based on the total weight of the particle. In another preferred embodiment, each particle contains odevixibat, or a pharmaceutically acceptable salt thereof, in an amount of about 1.0% w/w based on the total weight of the particle. In yet 3another preferred embodiment, each particle contains odevixibat, or a pharmaceutically acceptable salt thereof, in an amount of about 1.5% w/w based on the total weight of the particle.

As used herein, the term “particles” refers to small particles ranging in size from about 0.1 to about 1.5 mm. Such particles are preferably essentially spherical, although elongated or oblong particles also might be used. The particles may e.g. be pellets, beads, microparticles, microspheres, granules or minitablets, and may optionally be coated with one or more coating layers surrounding every such pellet, bead, microparticle, microsphere, granule or minitablet.

In some embodiments, the particles of the formulation are small enough, that they can be sprinkled onto food and easily swallowed. In some embodiments, the particles can be swallowed without causing a perception of grittiness. In some embodiments, the particles do not give the patient an urge to chew the particles. The particles are, therefore, preferably between about 0.1 and about 1.5 mm in size, more preferably between about 0.1 and about 1.0 mm, and more preferably between about 0.1 and 0.8 mm, such as about 0.2 mm, about 0.3 mm, about 0.4 mm, about 0.5 mm, about 0.6 mm, or about 0.7 mm. In a more preferred embodiment, the particles are between about 0.4 and about 0.8 mm, such as about 0.5 mm, or such as about 0.6 mm, or such as about 0.7 mm. In a particular embodiment of the invention, the particles are about 0.7 mm.

In some embodiments, the invention relates to a formulation of odevixibat, wherein each particle comprises a core and a coating layer surrounding the core. The core of each particle may be a pellet, a granule, a minitablet, a bead, a microparticle or a microsphere.

In some embodiments, the core of each particle comprises the active pharmaceutical ingredient (odevixibat), while the coating layer of each particle does not comprise the active pharmaceutical ingredient. In some embodiments, the core of each particle comprises from about 0.1% to about 5% w/w of the active pharmaceutical ingredient, based on the total weight of the particle, such as from about 0.1% to about 2% w/w, such as from about 0.1% to about 1% w/w, or such as from about 0.1% to about 0.5% w/w of the active pharmaceutical ingredient, based on the total weight of the particle.

In some embodiments, the coating layer of each particle comprises the active pharmaceutical ingredient (odevixibat), while the core of each particle does not comprise the active pharmaceutical ingredient. In some embodiments, the coating layer of each particle comprises from about 0.1% to about 5% w/w of the active pharmaceutical ingredient, based on the total weight of the particle, such as from about 0.1% to about 2% w/w, such as from about 0.1% to about 1% w/w, or such as from about 0.1% to about 0.5% w/w of the active pharmaceutical ingredient, based on the total weight of the particle.

The cores may be orally dispersible and comprise soluble ingredients such as a sugar (e.g., sucrose) or a soluble polymer (e.g. hydroxypropyl methylcellulose) or may be non-orally dispersible and comprise non-soluble ingredients such as a non-soluble polymer (e.g., microcrystalline cellulose). In a preferred embodiment of the invention, the cores comprise microcrystalline cellulose. In a more preferred embodiment, the cores are microcrystalline cellulose spheres.

The coating layer can further comprise a film-forming polymer, such as a cellulose-based polymer, a polysaccharide-based polymer, an N-vinylpyrrolidone-based polymer, an acrylate, an acrylamide, or copolymers thereof. Examples of suitable film-forming polymers include polyvinyl alcohol (PVA), polyvinyl acetate phthalate (PVAP), polyethylene glycol (PEG), polyvinylpyrrolidone (PVP), methacrylic acid copolymers, starch, hydroxypropyl starch, chitosan, shellac, methyl cellulose, hydroxypropyl cellulose (HPC), low-substituted hydroxypropyl cellulose, hydroxypropyl methylcellulose (HPMC; or hypromellose), hydroxypropyl methylcellulose acetate succinate (HPMCAS), hydroxypropyl methylcellulose phthalate (HPMCP), cellulose acetate phthalate (CAP), cellulose acetate trimellitate (CAT), as well as combinations thereof, such as a mixture of methyl cellulose and hydroxypropyl methylcellulose (metolose). In a preferred embodiment, the coating layer comprises a film-forming polymer selected from the group consisting of hydroxypropyl methylcellulose, polyvinyl alcohol (PVA), polyethylene glycol (PEG), starch, hydroxypropyl starch and hydroxypropyl cellulose (HPC). In a most preferred embodiment, the coating layer comprises hydroxypropyl methylcellulose as the film-forming polymer.

The coating layer may optionally comprise one or more additional ingredients, such as a plasticizer (e.g. polyethylene glycol, triacetin or triethyl citrate), an anti-tack agent (e.g. talc or magnesium stearate) or a colouring agent (e.g. titanium dioxide, iron oxides, riboflavin or turmeric).

In some embodiments, the formulation comprises odevixibat in crystalline form. In some embodiments, the formulation comprises a crystalline hydrate of odevixibat. In some embodiments, the formulation comprises crystal modification 1 of odevixibat. This stable crystal modification can be obtained from a slurry of odevixibat in a mixture of water and an organic solvent such as ethanol. Under these conditions, a mixed solvate containing about two moles of water and about one to about three, such as about two to about three, moles of ethanol per mole of odevixibat (e.g., a dihydrate-diethanolate or a dihydrate-triethanolate) is initially formed. This mixed solvate is referred to herein as crystal modification 2. When crystal modification 2 is dried, such as under vacuum (e.g., less than 5 mbar) or under a nitrogen flow, it loses its organic solvent molecules and becomes crystal modification 1. In some embodiments, the transformation of crystal modification 2 to crystal modification 1 proceeds via a crystalline intermediate. It is believed that this crystalline intermediate is a dehydrated form, which quickly takes up water from the air. While not wishing to be bound by theory, it is believed that the solvent molecules can be removed without dissolution and recrystallization of the crystals.

Crystal modification 1 of odevixibat cannot only be obtained from a mixture of water and ethanol, as described above, but also from a slurry of odevixibat in a mixture of water and an organic solvent selected from the group consisting of methanol, 2-propanol, acetone, acetonitrile, 1,4-dioxane, DMF and DMSO. Upon drying of the different mixed solvates obtained under these conditions (crystal modification 2), the same crystalline hydrate of odevixibat is obtained, namely crystal modification 1.

Crystal modification 1 contains void volumes that are capable of containing up to about 2 moles of water associated with the crystal per mole of odevixibat, depending on the relative humidity. This form is therefore formally a channel hydrate. At about 30% relative humidity, however, crystal modification 1 contains a substantially stoichiometric amount of about 1.5 moles of water per mole of organic compound and is thus a sesquihydrate. The substantially stoichiometric amount of water is considered advantageous, as the water content of the crystals remains substantially constant even with humidity changes within the normal relative humidity range of about 30% to about 70% RH. Indeed, at normal humidities, such as between about 30 and about 70% RH, crystal modification 1 exhibits relatively low hygroscopicity.

In one embodiment, the formulation comprises crystal modification 1 of odevixibat having an X-ray powder diffraction (XRPD) pattern, obtained with CuKα1-radiation, with at least specific peaks at °2θ positions 5.6±0.2, 6.7±0.2 and/or 12.1±0.2.

In a specific embodiment, the formulation comprises crystal modification 1 having an XRPD pattern, obtained with CuKα1-radiation, with specific peaks at °2θ positions 5.6±0.2, 6.7±0.2 and 12.1±0.2 and one or more of the characteristic peaks: 4.1±0.2, 4.6±0.2, 9.3±0.2, 9.4±0.2 and 10.7±0.2.

In a more specific embodiment, the formulation comprises crystal modification 1 having an XRPD pattern, obtained with CuKα1-radiation, with specific peaks at °2θ positions 4.6±0.2, 5.6±0.2, 6.7±0.2, 9.3±0.2, 9.4±0.2 and 12.1±0.2.

In a more specific embodiment, the formulation comprises crystal modification 1 having an XRPD pattern, obtained with CuKα1-radiation, with characteristic peaks at °2θ positions 4.1±0.2, 4.6±0.2, 5.6±0.2, 6.7±0.2, 9.3±0.2, 9.4±0.2, 10.7±0.2 and 12.1±0.2, and one or more of 8.1±0.2, 8.6±0.2, 13.4±0.2, 13.8±0.2, 13.9±0.2, 16.6±0.2, 17.3±0.2, 17.7±0.2, 18.3±0.2, 18.9±0.2, 19.4±0.2, 19.7±0.2, 20.5±0.2, 20.8±0.2, 21.6±0.2, 23.2±0.2, 24.3±0.2, 29.8±0.2 and 30.6±0.2.

In an even more specific embodiment, the formulation comprises crystal modification 1 having an XRPD pattern, obtained with CuKα1-radiation, with characteristic peaks at °2θ positions 4.1±0.2, 4.6±0.2, 5.6±0.2, 6.7±0.2, 8.1±0.2, 8.6±0.2, 9.3±0.2, 9.4±0.2, 10.7±0.2, 12.1±0.2, 13.4±0.2, 13.8±0.2, 13.9±0.2, 16.6±0.2, 17.3±0.2, 17.7±0.2, 18.3±0.2, 18.9±0.2, 19.4±0.2, 19.7±0.2, 20.5±0.2, 20.8±0.2, 21.6±0.2, 23.2±0.2, 24.3±0.2, 29.8±0.2 and 30.6±0.2.

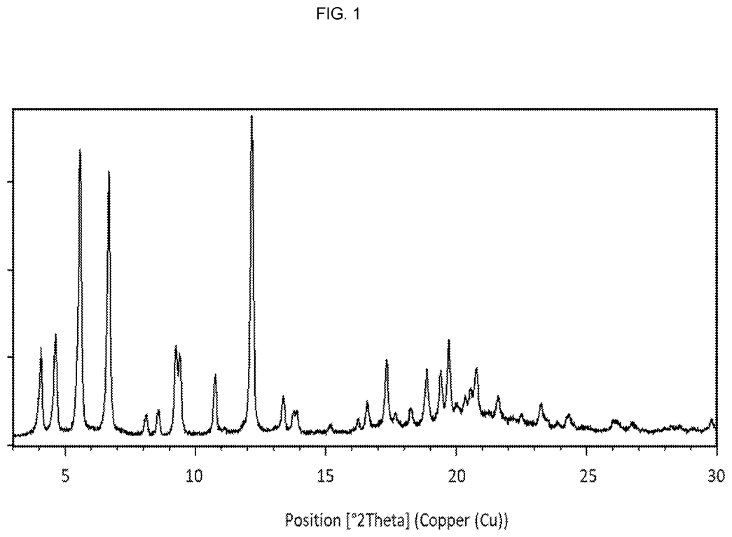

In another embodiment, the formulation comprises crystal modification 1 having an XRPD pattern, obtained with CuKα1-radiation, substantially as shown in FIG. 1.

Whereas crystal modification 1 is a sesquihydrate containing about 3.5% (w/w) water at about 30% relative humidity (based on the total crystal weight), it has been observed that the crystal can take up an additional 1.5% (w/w) water when the humidity is increased up to 95% RH. The sorption and desorption of this additional water is fully reversible. The additional water may be adsorbed on the surface or may further fill the channels of the structure. In some embodiments, the term “overhydrated” refers to crystal modification 1 containing from about 1.5 to about 4 moles of water per mole of odevixibat, such as from about 1.5 to about 3.5, or such as from about 1.5 to 3, or such as from about 1.5 to about 2.5, or such as from about 1.5 to about 2 moles of water per mole of odevixibat. In some embodiments, the term “overhydrated” refers to crystal modification 1 containing from about 2 to about 4 moles of water per mole of odevixibat, such as from about 2 to about 3.5, or such as from about 2 to about 3, or such as from about 2 to 2.5 moles of water per mole of odevixibat.

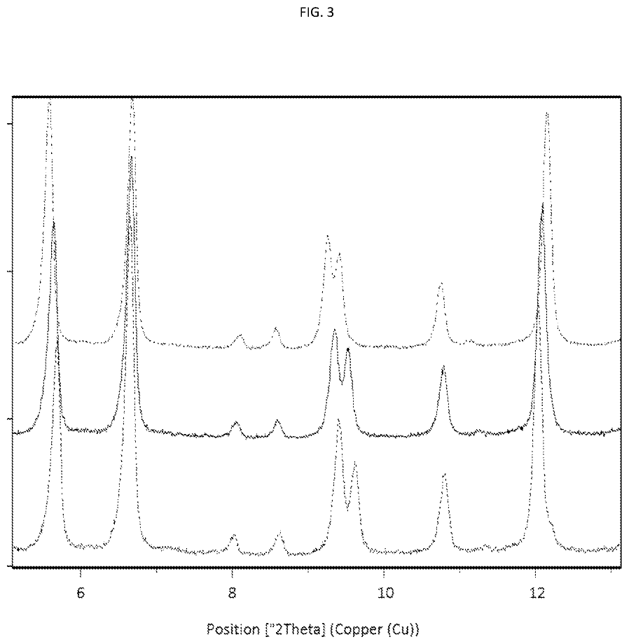

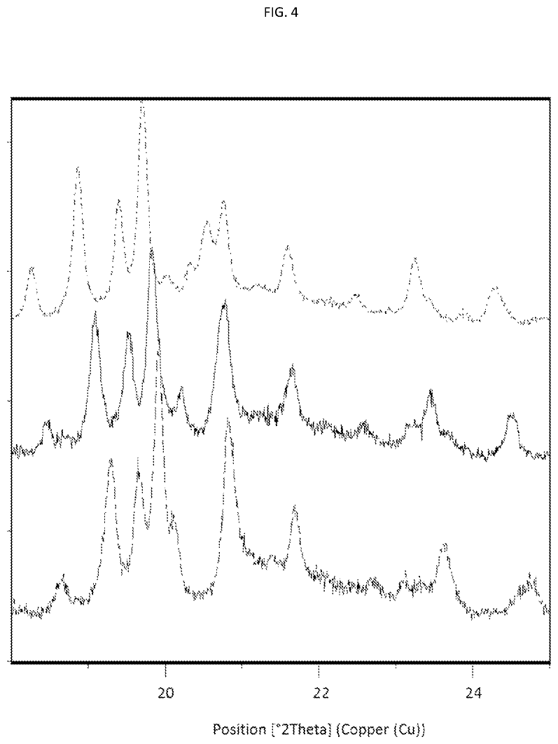

It has been observed that the XRPD pattern of overhydrated crystal modification 1 slightly changes when it is dried, e.g. at 50° C. in vacuum. A small shift of peaks is most clearly seen in the 2θ ranges 5-13° and 18-25°, as shown in FIGS. 3 and 4, respectively. Exposing the dried modification to elevated relative humidity, such as up to 95% RH, makes the XRPD pattern of the overhydrated modification appear again. The peak shifts are a result of the unit cell volume changes, which occur as water molecules go in and out of the crystal structure.

Therefore, in another embodiment, the formulation comprises overhydrated crystal modification 1 having an X-ray powder diffraction (XRPD) pattern, obtained with CuKα1-radiation, with at least specific peaks at °2θ positions 5.7±0.2, 6.7±0.2 and/or 12.0±0.2.

In a specific embodiment, the formulation comprises overhydrated crystal modification 1 having an XRPD pattern, obtained with CuKα1-radiation, with specific peaks at °2θ positions 5.7±0.2, 6.7±0.2 and 12.0±0.2 and one or more of the characteristic peaks: 4.0±0.2, 9.4±0.2, 9.6±0.2 and 10.8±0.2.

In a more specific embodiment, the formulation comprises overhydrated crystal modification 1 having an XRPD pattern, obtained with CuKα1-radiation, with specific peaks at °2θ positions 4.0±0.2, 5.7±0.2, 6.7±0.2, 9.4±0.2, 9.6±0.2, 10.8±0.2 and 12.1±0.2.

In a more specific embodiment, the formulation comprises overhydrated crystal modification 1 having an XRPD pattern, obtained with CuKα1-radiation, with characteristic peaks at °2θ positions 4.0±0.2, 5.7±0.2, 6.7±0.2, 9.4±0.2, 9.6±0.2, 10.8±0.2 and 12.1±0.2, and one or more of 4.7±0.2, 8.0±0.2, 8.6±0.2, 13.3±0.2, 14.1±0.2, 15.3±0.2, 16.5±0.2, 17.3±0.2, 19.3±0.2, 19.7±0.2, 19.9±0.2, 20.1±0.2, 20.8±0.2, 21.7±0.2, 23.6±0.2, 26.2±0.2, 26.5±0.2, 28.3±0.2 and 30.9±0.2.

In an even more specific embodiment, the formulation comprises overhydrated crystal modification 1 having an XRPD pattern, obtained with CuKα1-radiation, with characteristic peaks at °2θ positions 4.0±0.2, 4.7±0.2, 5.7±0.2, 6.7±0.2, 8.0±0.2, 8.6±0.2, 9.4±0.2, 9.6±0.2, 10.8±0.2, 12.1±0.2, 13.3±0.2, 14.1±0.2, 15.3±0.2, 16.5±0.2, 17.3±0.2, 19.3±0.2, 19.7±0.2, 19.9±0.2, 20.1±0.2, 20.8±0.2, 21.7±0.2, 23.6±0.2, 26.2±0.2, 26.5±0.2, 28.3±0.2 and 30.9±0.2.

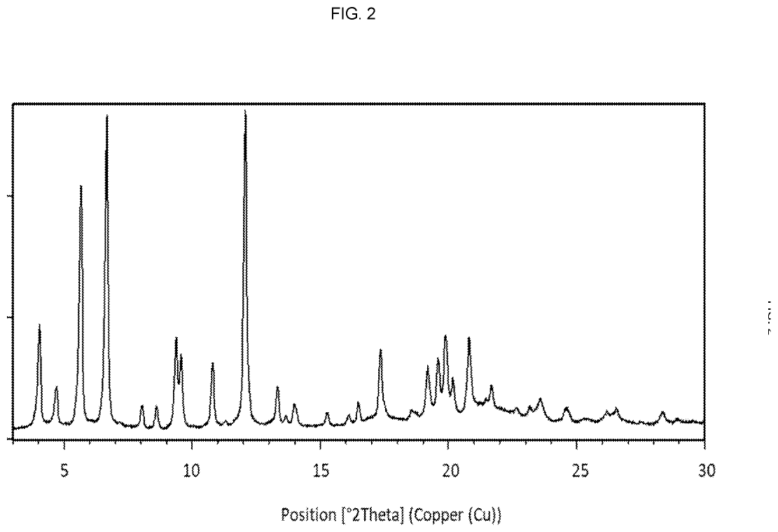

In another embodiment, the formulation comprises overhydrated crystal modification 1 of odevixibat having an XRPD pattern, obtained with CuKα1-radiation, substantially as shown in FIG. 2.

It is desirable that the use of organic solvents in the preparation of the formulation is avoided. In some embodiments, water is used as the solvent for the preparation of the formulation. Odevixibat dissolves in water only very poorly, and the solubility at pH 7 and at 37° C. has been determined to be as low as about 30 μg/mL. Because of this low solubility in water, aqueous suspensions of odevixibat can contain larger agglomerates of odevixibat, which may lead to an uneven distribution of the active pharmaceutical ingredient on the cores, i.e. the cores may contain different amounts of odevixibat, which in turn impacts dose uniformity. Accordingly, in some embodiments, the aqueous suspension of odevixibat is homogeneous. In some embodiments, a homogeneous aqueous suspension of odevixibat is sprayed onto the cores.

Odevixibat exhibits high potency and it should be administered in low doses, especially in the treatment of pediatric patients that weigh about 5 to 20 kg. In order to reach high dose uniformity for the multiparticulate formulation disclosed herein, it is important that each particle of the formulation substantially contains the same amount of odevixibat, i.e., the deviation in the odevixibat content of the particles of the formulation should be as low as possible.

As used herein, the term “homogeneous” refers to a suspension that does not contain agglomerates of odevixibat that are larger than about 200 μm, and preferably no agglomerates larger than about 100 μm, more preferably no agglomerates larger than about 50 μm. The size of the odevixibat agglomerates in the coating suspension may be determined by optical microscopy, using a method based on European Pharmacopoeia 9.0, monograph 2.9.37, and as described in the experimental section. Alternatively, the size of the odevixibat agglomerates in the coating suspension may be determined by light scattering techniques, such as low-angle laser light scattering (LALLS). In some embodiments, the d₉₀ value for the particle size distribution of the coating suspension is smaller than 15 μm, such as smaller than 14 μm, such as smaller than 13 μm, such as smaller than 12 μm, such as smaller than 11 μm, or such as smaller than 10 μm.

In some embodiments, a homogeneous suspension of odevixibat can be prepared by dispersing the compound in water by wet-milling. Wet-milling is a process in which a solid substance is dispersed in a liquid by shearing, by crushing, or by attrition. Examples of wet-milling apparatus include colloid mills, conical mills, ball mills, disc mills and high-shear dispersing machines. A specific example of a wet-milling apparatus for use in the present invention is a colloid mill.

In some embodiments, the crystallinity of odevixibat increases during the wet-milling.

Preferably, odevixibat is first wetted in a small amount of water using a homogenizer and thereafter dispersed in water using a colloid mill. Spraying the homogenized dispersion onto the cores enables an even distribution of the active pharmaceutical ingredient.

It is desirable that the formulation is free of any ingredients that are not strictly necessary for the formulation, such as surfactants. In a preferred embodiment, therefore, the coating suspension does not contain surfactants. Similarly, in some embodiments, the coating layer of the formulation does not contain surfactants.

In one embodiment, the particles are contained within a sachet. In another embodiment, the particles are contained within a capsule. Such capsules may be made from gelatine, from a cellulose-based polymer such as a hydroxypropyl methylcellulose (hypromellose), or from a polysaccharide-based polymer such as a pullulan. Capsules may be swallowed intact, or may be designed to be opened, so that, for example, the contents (i.e. the particles) can be sprinkled onto a food vehicle for administration. In the latter case, the number of particles in one capsule should preferably fit onto a single tablespoon of food. In some embodiments, a capsule contains from about 20 to about 100 mg of particles, such as about 30, about 40, about 50, about 60, about 70, about 80 or about 90 mg.

For younger paediatric patients, such as infants, toddlers and children up to about 6 years old, the particles are preferably sprinkled onto food that can be easily swallowed and which does not require chewing, such as yoghurt, apple sauce, fruit purée or oatmeal. For older paediatric patients, such as children older than about 6 years old, adolescents and younger adults, capsules containing the particles may be swallowed intact, i.e. without opening. For newborn patients up to about 6 months old, who have not yet been weaned or are unable to take semi-solid food, the formulation can be administered by dispersing the particles in a suitable liquid vehicle, such as breast milk, baby formula or water. When the particles have been dispersed in a liquid vehicle, they can be administered to the patient within 30 minutes after dispersion, without loss of the active ingredient or indications of degradation. In some embodiments, the volume of liquid vehicle used for administering the odevixibat particles, including rinsing, can be smaller than about 20 mL, such as smaller than about 15 mL, such as smaller than about 10 mL, or such as smaller than about 5 mL. In some embodiments, the dispersed particles are administered directly into the mouth using an oral syringe.

The formulation disclosed herein may be used in the treatment or prevention of liver diseases, such as bile acid-dependent liver diseases. In some embodiments, a liver disease involves elevated levels of bile acids in the serum and/or in the liver. The formulation disclosed herein may in particular be used in the treatment or prevention of cholestatic liver diseases, including rare paediatric cholestatic liver diseases, such as biliary atresia; post-Kasai biliary atresia; post-liver transplantation biliary atresia; progressive familial intrahepatic cholestasis (PFIC), including PFIC-1, PFIC-2, PFIC-3 and non-specified PFIC, post-biliary diversion PFIC and post-liver transplant PFIC; Alagille syndrome (ALGS); and primary biliary cirrhosis (PBC); as well as paediatric cholestatic pruritus. In one aspect, therefore, the invention relates to the formulation disclosed herein for use in the treatment or prevention of a cholestatic liver disease. In another aspect, the invention relates to a method of treating or preventing a cholestatic liver disease in a subject, such as a human, comprising administering to the subject in need of such treatment or prevention a therapeutically effective amount of the formulation disclosed herein.

Biliary atresia is a rare pediatric liver disease that involves a partial or total blockage (or even absence) of large bile ducts. This blockage or absence causes cholestasis that leads to the accumulation of bile acids that damages the liver. In some embodiments, the accumulation of bile acids occurs in the extrahepatic biliary tree. In some embodiments, the accumulation of bile acids occurs in the intrahepatic biliary tree. The current standard of care is the Kasai procedure, which is a surgery that removes the blocked bile ducts and directly connects a portion of the small intestine to the liver. There are currently no approved drug therapies for this disorder.

Provided herein are methods for treating biliary atresia in a subject in need thereof, the methods comprising administration of a therapeutically effective amount of the formulation disclosed herein. In some embodiments, the subject has undergone the Kasai procedure prior to administration of the formulation disclosed herein. In some embodiments, the subject is administered the formulation disclosed herein prior to undergoing the Kasai procedure. In some embodiments, the treatment of biliary atresia decreases the level of serum bile acids in the subject. In some embodiments, the level of serum bile acids is determined by, for example, an ELISA enzymatic assay or the assays for the measurement of total bile acids as described in Danese et al., PLoS One. 2017, vol. 12(6):e0179200, which is incorporated by reference herein in its entirety. In some embodiments, the level of serum bile acids can decrease by, for example, 10% to 40%, 20% to 50%, 30% to 60%, 40% to 70%, 50% to 80%, or by more than 90% of the level of serum bile acids prior to administration of the formulation disclosed herein. In some embodiments, the treatment of biliary atresia includes treatment of pruritus.

PFIC is a rare genetic disorder that is estimated to affect between one in every 50,000 to 100,000 children born worldwide and causes progressive, life-threatening liver disease.

One manifestation of PFIC is pruritus, which often results in a severely diminished quality of life. In some cases, PFIC leads to cirrhosis and liver failure. Current therapies include Partial External Biliary Diversion (PEBD) and liver transplantation, however, these options can carry substantial risk of post-surgical complications, as well as psychological and social issues.

Three alternative gene defects have been identified that correlate to three separate PFIC subtypes known as types 1, 2 and 3.
PFIC, type 1, which is sometimes referred to as “Byler disease,” is caused by impaired bile secretion due to mutations in the ATP8B1 gene, which codes for a protein that helps to maintain an appropriate balance of fats known as phospholipids in cell membranes in the bile ducts. An imbalance in these phospholipids is associated with cholestasis and elevated bile acids in the liver. Subjects affected by PFIC, type 1 usually develop cholestasis in the first months of life and, in the absence of surgical treatment, progress to cirrhosis and end-stage liver disease before the end of the first decade of life.

PFIC, type 2, which is sometimes referred to as “Byler syndrome,” is caused by impaired bile salt secretion due to mutations in the ABCB11 gene, which codes for a protein, known as the bile salt export pump, that moves bile acids out of the liver. Subjects with PFIC, type 2 often develop liver failure within the first few years of life and are at increased risk of developing a type of liver cancer known as hepatocellular carcinoma.

PFIC, type 3, which typically presents in the first years of childhood with progressive cholestasis, is caused by mutations in the ABCB4 gene, which codes for a transporter that moves phospholipids across cell membranes.

In addition, TJP2 gene, NR1H4 gene or Myo5b gene mutations have been proposed to be causes of PFIC. In addition, some subjects with PFIC do not have a mutation in any of the ATP8B1, ABCB11, ABCB4, TJP2, NR1H4 or Myo5b genes. In these cases, the cause of the condition is unknown.

Exemplary mutations of the ATP8B1 gene or the resulting protein are listed in Tables 1 and 2, with numbering based on the human wild type ATP8B1 protein (e.g., SEQ ID NO: 1) or gene (e.g., SEQ ID NO: 2). Exemplary mutations of the ABCB11 gene or the resulting protein are listed in Tables 4 and 5, with numbering based on the human wild type ABCB11 protein (e.g., SEQ ID NO: 3) or gene (e.g., SEQ ID NO: 4).

As can be appreciated by those skilled in the art, an amino acid position in a reference protein sequence that corresponds to a specific amino acid position in SEQ ID NO: 1 or 3 can be determined by aligning the reference protein sequence with SEQ ID NO: 1 or 3 (e.g., using a software program, such as ClustalW2). Changes to these residues (referred to herein as “mutations”) may include single or multiple amino acid substitutions, insertions within or flanking the sequences, and deletions within or flanking the sequences. As can be appreciated by those skilled in the art, an nucleotide position in a reference gene sequence that corresponds to a specific nucleotide position in SEQ ID NO: 2 or 4 can be determined by aligning the reference gene sequence with SEQ ID NO: 2 or 4 (e.g., using a software program, such as ClustalW2). Changes to these residues (referred to herein as “mutations”) may include single or multiple nucleotide substitutions, insertions within or flanking the sequences, and deletions within or flanking the sequences. See also Kooistra, et al., “KLIFS: A structural kinase-ligand interaction database,” Nucleic Acids Res. 2016, vol. 44, no. D1, pp. D365-D371, which is incorporated by reference in its entirety herein.

TABLE 1
Exemplary ATP8B1 Mutations

Amino acid position 3 (e.g., T3K)²⁷
Amino acid position 23 (e.g., P23L)⁵
Amino acid position 45 (e.g., N45T)^{5,8,9}
Amino acid position 46 (e.g., R46X)^{A,25}
Amino acid position 62 (e.g., C62R)²⁸
Amino acid position 63 (e.g., T63T)⁴¹
Amino acid position 70 (e.g., D70N)^{1,6}
Amino acid position 71 (e.g., R71H)⁴³
Amino acid position 78 (e.g., H78Q)¹⁹
Amino acid position 82 (e.g., T82T)⁴¹
Amino acid position 92 (e.g., Y92Y)⁴¹
Amino acid position 93 (e.g., A93A)⁶
Amino acid position 96 (e.g., A96G)²⁷
Amino acid position 114 (e.g., E114Q)⁸
Amino acid position 127 (e.g., L127P⁶, L127V³⁶)
Amino acid position 177 (e.g., T177T)⁶
Amino acid position 179 (e.g., E179X)²⁹
Δ Amino acid positions 185-282⁴⁴
Amino acid position 197 (e.g., G197Lfs*10)²²
Amino acid position 201 (e.g., R201S²⁷, R201H³⁵)
Amino acid position 203 (e.g., K203E^{5,8}, K203R⁹, K203fs²⁵)
Amino acid position 205 (e.g., N205fs⁶, N205Kfs*2³⁵)
Amino acid position 209 (e.g., P209T)⁴
Amino acid position 217 (e.g., S217N)⁴³
Amino acid position 232 (e.g., D232D)³⁰
Amino acid position 233 (e.g., G233R)³⁸
Amino acid position 243 (e.g., L243fs*28)³³
Amino acid position 265 (e.g., C265R)²⁵
Amino acid position 271 (e.g., R271X¹³, R271R³⁰)
Amino acid position 288 (e.g., L288S)⁶
Amino acid position 294 (e.g., L294S)⁴³
Amino acid position 296 (e.g., R296C)¹¹
Amino acid position 305 (e.g., F305I)²⁸
Amino acid position 306 (e.g., C306R)²³
Amino acid position 307 (e.g., H307L)³⁵
Amino acid position 308 (e.g., G308V¹, G308D⁶, G308S³⁵)
Amino acid position 314 (e.g., G314S)¹³
Amino acid position 320 (e.g., M320Vfs*13)¹¹
Amino acid position 337 (e.g., M337R)¹⁸
Amino acid position 338 (e.g., N338K)¹⁸
Amino acid position 340 (e.g., M340V)¹⁸
Amino acid position 344 (e.g., I344F)^{6,20}
Amino acid position 349 (e.g., I349T)⁴¹
Amino acid position 358 (e.g., G358R)²⁸
Amino acid position 367 (e.g., G367G)⁴¹
Amino acid position 368 (e.g., N368D)⁴¹
Amino acid position 393 (e.g., I393V)²⁷
Amino acid position 403 (e.g., S403Y)⁶
Amino acid position 407 (e.g., S407N)⁴⁰
Amino acid position 412 (e.g., R412P)⁶
Amino acid position 415 (e.g., Q415R)²⁷
Amino acid position 422 (e.g., D422H)³⁵
Amino acid position 429 (e.g., E429A)⁶
Amino acid position 446 (e.g., G446R)^{4,11}
Amino acid position 453 (e.g., S453Y)⁶
Amino acid position 454 (e.g., D454G)⁶
Amino acid position 455 (e.g., K455N)⁴³
Amino acid position 456 (e.g., T456M^{3,6}, T456K³⁵)
Amino acid position 457 (e.g., G457G⁶, G457fs*6³³)
Amino acid position 469 (e.g., C469G)⁴¹
Amino acid position 478 (e.g., H478H)⁴¹
Amino acid position 500 (e.g., Y500H)⁶
Amino acid position 525 (e.g., R525X)⁴
Δ Amino acid position 529⁶
Amino acid position 535 (e.g., H535L⁶, H535N⁴¹)
Amino acid position 553 (e.g., P553P)⁴³
Amino acid position 554 (e.g., D554N^{1,6}, D554A³⁵)
Δ Amino acid positions 556-628⁴⁴
Δ Amino acid positions 559-563³⁵
Amino acid position 570 (e.g., L570L)⁴¹
Amino acid position 577 (e.g., I577V)¹⁹
Amino acid position 581 (e.g., E581K)³⁵
Amino acid positions 554 and 581 (e.g., D554A+E581K)³⁵
Amino acid position 585 (e.g., E585X)²¹
Amino acid position 600 (e.g., R600W^{2,4}, R600Q⁶)
Amino acid position 602 (e.g., R602X)^{3,6}
Amino acid position 628 (e.g., R628W)⁶
Amino acid position 631 (e.g., R631Q)²⁸
Δ Amino acid positions 645-699⁴
Amino acid position 661 (e.g., I661T)^{1,4,6}
Amino acid position 665 (e.g., E665X)^{4,6}
Amino acid position 672 (e.g., K672fs⁶, K672Vfs*1³⁵)
Amino acid position 674 (e.g., M674T)¹⁹
Amino acid positions 78 and 674 (e.g., H78Q/M674T)¹⁹
Amino acid position 684 (e.g., D684D)⁴¹
Amino acid position 688 (e.g., D688G)⁶
Amino acid position 694 (e.g., I694T⁶, I694N¹⁷)
Amino acid position 695 (e.g., E695K)²⁷
Amino acid position 709 (e.g., K709fs⁶, K709Qfs*41¹³)
Amino acid position 717 (e.g., T717N)⁴
Amino acid position 733 (e.g., G733R)⁶
Amino acid position 757 (e.g., Y757X)⁴
Amino acid position 749 (e.g., L749P)²¹
Amino acid position 792 (e.g., P792fs)⁶
Δ Amino acid position 795-797⁶
Amino acid position 809 (e.g., I809L)²⁷
Amino acid position 814 (e.g., K814N)²⁸
Amino acid position 833 (e.g., R833Q²⁷, R833W⁴¹)
Amino acid position 835 (e.g., K835Rfs*36)³⁵
Amino acid position 845 (e.g., K845fs)²⁵
Amino acid position 849 (e.g., R849Q)²⁴
Amino acid position 853 (e.g., F853S, F853fs)⁶
Amino acid position 867 (e.g., R867C¹, R867fs⁶, R867H²³)
Amino acid position 885 (e.g., K885T)⁴¹
Amino acid position 888 (e.g., T888T)⁴¹
Amino acid position 892 (e.g., G892R)⁶
Amino acid position 912 (e.g., G912R)³⁵
Amino acid position 921 (e.g., S921S)⁴¹
Amino acid position 924 (e.g., Y924C)²⁸
Amino acid position 930 (e.g., R930X⁶, R930Q²⁸)
Amino acid position 941 (e.g., R941X)³⁵
Amino acid position 946 (e.g., R946T)⁴¹
Amino acid position 952 (e.g., R952Q^{5,9,15}, R952X⁶)
Amino acid position 958 (e.g., N958fs)⁶
Amino acid position 960 (e.g., A960A)⁴¹
Δ Amino acid position 971⁴³
Amino acid position 976 (e.g., A976E⁴¹, A976A⁴³)
Amino acid position 981 (e.g., E981K)²⁰
Amino acid position 994 (e.g., S994R)⁴
Amino acid position 1011 (e.g., L1011fs*18)³³
Amino acid position 1012 (e.g., S1012I)¹⁰
Amino acid position 1014 (e.g., R1014X)^{6,11}
Amino acid position 1015 (e.g., F1015L)²⁷
Amino acid position 1023 (e.g., Q1023fs)⁶
Amino acid position 1040 (e.g., G1040R)^{1,6}
Amino acid position 1044 (e.g., S0144L)³⁴
Amino acid position 1047 (e.g., L1047fs)⁶
Amino acid position 1050 (e.g., I1050K)³¹
Amino acid position 1052 (e.g., L1052R)²⁸
Amino acid position 1095 (e.g., W1095X)¹¹
Amino acid position 1098 (e.g., V1098X)³⁵
Amino acid position 1131 (e.g., Q1131X)⁴⁴
Amino acid position 1142 (e.g., A1142Tfs*35)⁴³
Amino acid position 1144 (e.g., Y1144Y)⁴³
Amino acid position 1150 (e.g., I1150T)⁴¹
Amino acid position 1152 (e.g., A1152T)³⁰
Amino acid position 1159 (e.g., P1159P)^{25,43}
Amino acid position 1164 (e.g., R1164X)⁶
Amino acid position 1193 (e.g., R1193fs*39)³³
Amino acid position 1197 (e.g., V1197L)⁴¹
Amino acid position 1208 (e.g., A1208fs)⁶
Amino acid position 1209 (e.g., Y1209Lfs*28)⁴
Amino acid position 1211 (e.g., F1211L)²⁷
Amino acid position 1219 (e.g., D1219H⁵, D1219G²⁷)
Amino acid position 1223 (e.g., S1223S)⁴¹
Amino acid position 1233 (e.g., P1233P)⁴¹
Amino acid position 1241 (e.g., G1241fs)⁶
Amino acid position 1248 (e.g., T1248T)⁴³
Splice site mutation IVS3+1_+3delGTG⁶
Splice site mutation IVS3−2A>G⁶
IVS6+5T>G^{17,25}
Splice site mutation IVS8+1G>T⁶
IVS9−G>A²⁶
IVS12+1G>A²⁵
Splice site mutation IVS17−1G>A⁶
Splice site mutation IVS18+2T>C⁶
Splice site mutation IVS20−4CT>AA
Splice site mutation IVS21+5G>A⁶
Splice site mutation IVS23−3C>A⁶
Splice site mutation IVS26+2T>A⁶
g.24774-42062del⁴
c.−4C>G⁴¹
c.145C>T¹²
c.181−72G>A⁹
c.182−5T>A⁴¹
c.182−72G>A⁴¹
c.246A>G⁹
c.239G>A³⁹
c.279+1_279+3delGTG⁴⁶
c.280−2A>G⁴⁶
c.625_62715delinsACAGTAAT⁴⁶
c.554+122C>T⁹
c.555−3T>C²⁷
c.625+5 G>T⁴
Amino acid position 209 (e.g., P209T) and c.625+5 G>T⁴
c.628−30G>A⁴¹
c.628−31C>T⁴¹
c.698+1G>T⁴⁶
c.698+20C>T⁴¹
c.782−1G>A⁴⁶
c.782−34G>A⁴¹
Δ795-797¹⁴
c.782−1G>A⁴
c.852A>C²⁷
c.941−1G>A⁴⁶
c.1014C>T⁹
c.1029+35G>A⁹
c.1221−8C.G⁴¹
1226delA¹⁶
c.1429+1G>A⁴⁶
c.1429+2T>G¹³
c.1429+49G>A⁴¹
c.1430−42A>G⁴¹
c.1493T>C¹²
c.1587_1589delCTT⁴⁶
c.1630+2T>G²⁷
c.1631−10T>A⁴¹
c.1637−37T>C⁴¹
1660 G>A¹⁴
1798 C>T¹⁴
1799 G>A¹⁴
c.1819−39_41delAA⁹
c.1819+1G>A³¹
c.1820−27G>A⁴¹
c.1918+8C>T²⁷
c.1933−1G>AK46
c.2097+2T>C³²
c.2097+60T>G⁴¹
c.2097+89T>C⁴¹
c.2097+97T>G⁴¹
c.2210−114T>C⁹
2210delA¹⁶
c.2210−45_50dupATAAAA⁹
c.2285+29C.T⁴¹
c.2285+32A>G⁴¹
c.2286−4_2286-3delinsAA⁴⁶
c.2418+5G>A⁴⁶
c.2707+3G>C²⁷
c.2707+9T>G⁴¹
c.2707+43A>G⁴¹
c.2709−59T>C⁴¹
c.2931+9A>G⁴¹
c.2931+59T>A⁴¹
c.2932−3C>A⁴⁶
c.2932+59T>A⁹
c.2937A>C²⁷
c.3016−9C>A³¹
c.3033-3034del¹⁹
3122delTCCTA/insACATCGATGTTGATGTTAGG⁴⁵
3318 G>A¹⁴
c.3400+2T>A⁴⁶
c.3401−175C>T⁹
c.3401−167C>T⁹
c.3401−108C>T⁹
c.3531+8G>T^{9,15}
c.3532−15C>T⁹
Δ Phe ex 15⁴
Ex1_Ex13del⁶
Ex2_Ex6del³³
Ex12_Ex14del²⁷
Skipped Exon 24⁴⁵
del5′UTR-ex18¹¹
c.*11C>T⁴¹
c.*1101+366G>A⁷
g.92918del565³¹
GC preceding exon 16 (e.g., resulting in a 4 bp deletion)⁴²
Frameshift from the 5′ end of exon 16⁴²
5′ 1.4 kb deletion⁴⁶

TABLE 2
Selected ATP8B1 Mutations Associated with PFIC-1

Amino acid position 23 (e.g., P23L)⁵
Amino acid position 78 (e.g., H78Q)¹⁹
Amino acid position 93 (e.g., A93A)⁶
Amino acid position 96 (e.g., A96G)²⁷
Amino acid position 127 (e.g., L127P)⁶
Amino acid position 197 (e.g., G197Lfs*10)²²
Amino acid position 205 (e.g., N205fs)⁶
Amino acid position 209 (e.g., P209T)⁴
Amino acid position 233 (e.g., G233R)³⁸
Amino acid position 243 (e.g., L243fs*28)³³
Amino acid position 288 (e.g., L288S)⁶
Amino acid position 296 (e.g., R296C)¹¹
Amino acid position 308 (e.g., G308V^{1,6})
Amino acid position 320 (e.g., M320Vfs*13)¹¹
Amino acid position 403 (e.g., S403Y)⁶
Amino acid position 407 (e.g., S407N)⁴⁰
Amino acid position 412 (e.g., R412P)⁶
Amino acid position 415 (e.g., Q415R)²⁷
Amino acid position 429 (e.g., E429A)⁶
Amino acid position 446 (e.g., G446R)⁴
Amino acid position 456 (e.g., T456M)^{3,6}
Amino acid position 457 (e.g., G457G⁶, G457fs*6³³)
Amino acid position 500 (e.g., Y500H)⁶
Amino acid position 525 (e.g., R525X)⁴
Δ Amino acid position 529⁶
Amino acid position 535 (e.g., H535L)⁶
Amino acid position 554 (e.g., D554N)^{1,6}
Amino acid position 577 (e.g., I577V)¹⁹
Amino acid position 585 (e.g., E585X)²¹
Amino acid position 600 (e.g., R600W)⁴
Amino acid position 602 (e.g., R602X)^{3,6}
Amino acid position 661 (e.g., I661T)^{4,6}
Amino acid position 665 (e.g., E665X)^{4,6}
Δ Amino acid positions 645-699⁴
Amino acid position 672 (e.g., K672fs)⁶
Amino acid position 674 (e.g., M674T)¹⁹
Amino acid positions 78 and 674 (e.g., H78Q/M674T)¹⁹
Amino acid position 688 (e.g., D688G)⁶
Amino acid position 694 (e.g., I694N)¹⁷
Amino acid position 695 (e.g., E695K)²⁷
Amino acid position 709 (e.g., K709fs)⁶
Amino acid position 717 (e.g., T717N)⁴
Amino acid position 733 (e.g., G733R)⁶
Amino acid position 749 (e.g., L749P)²¹
Amino acid position 757 (e.g., Y757X)⁴
Amino acid position 792 (e.g., P792fs)⁶
Amino acid position 809 (e.g., I809L)²⁷
Amino acid position 853 (e.g., F853S, F853fs)⁶
Amino acid position 867 (e.g., R867fs)⁶
Amino acid position 892 (e.g., G892R)⁶
Amino acid position 930 (e.g., R930X⁶, R952Q¹⁵)
Amino acid position 952 (e.g., R952X)⁶
Amino acid position 958 (e.g., N958fs)⁶
Amino acid position 981 (e.g., E981K)²⁰
Amino acid position 994 (e.g., S994R)⁴
Amino acid position 1014 (e.g., R1014X)^{6,11}
Amino acid position 1015 (e.g., F1015L)²⁷
Amino acid position 1023 (e.g., Q1023fs)⁶
Amino acid position 1040 (e.g., G1040R)^{1,6}
Amino acid position 1047 (e.g., L1047fs)⁶
Amino acid position 1095 (e.g., W1095X)¹¹
Amino acid position 1208 (e.g., A1208fs)⁶
Amino acid position 1209 (e.g., Y1209Lfs*28)⁴
Amino acid position 1211 (e.g., F1211L)²⁷
Amino acid position 1219 (e.g., D1219H⁵, D1219G²⁷)
Splice site mutation IVS3+1_+3delGTG⁶
Splice site mutation IVS3−2A>G⁶
IVS6+5T>G¹⁷
Splice site mutation IVS8+1G>T⁶
IVS9−G>A²⁶
Splice site mutation IVS17−1G>A⁶
Splice site mutation IVS18+2T>C⁶
Splice site mutation IVS21+5G>A⁶
g.24774-42062del⁴
c.145C>T¹²
c.239G>A³⁹
c.625+5 G>T⁴
Amino acid position 209 (e.g., P209T) and c.625+5 G>T⁴
c.782−1G>A⁴
c.1493T>C¹²
c.1630+2T>G²⁷
1660 G>A¹⁴
c.2707+3G>C²⁷
c.2097+2T>C³²
c.3033-3034del¹⁹
3318 G>A¹⁴
c.3158+8G>T¹⁵
Δ Phe ex 15⁴
Ex1_Ex13del⁶
Ex2_Ex6del³³
Ex12_Ex14del²⁷
del5′UTR-ex18¹¹
c.*1101+366G>A⁷
GC preceding exon 16 (e.g., resulting in a 4 bp deletion)⁴²
Frameshift from the 5′ end of exon 16⁴²
^{A} A mutation to ′X′ denotes an early stop codon

### References for Tables 1 and 2

¹Folmer et al., Hepatology. 2009, vol. 50(5), p. 1597-1605.
²Hsu et al., Hepatol Res. 2009, vol. 39(6), p. 625-631.
³Alvarez et al., Hum Mol Genet. 2004, vol. 13(20), p. 2451-2460.
⁴Davit-Spraul et al., Hepatology 2010, vol. 51(5), p. 1645-1655.
⁵Vitale et al., J Gastroenterol. 2018, vol. 53(8), p. 945-958.
⁶Klomp et al., Hepatology 2004, vol. 40(1), p. 27-38.
⁷Zarenezhad et al., Hepatitis Monthly: 2017, vol. 17(2); e43500.
⁸Dixon et al., Scientific Reports 2017, vol. 7, 11823.
⁹Painter et al., Eur J Hum Genet. 2005, vol. 13(4), p. 435-439.
¹⁰ Deng et al., World J Gastroenterol. 2012, vol. 18(44), p. 6504-6509.
¹¹Giovannoni et al., PLoS One. 2015, vol. 10(12):e0145021.
¹²Li et al., Hepatology International 2017, vol. 11, No. 1, Supp. Supplement 1, pp. S180. Abstract Number: OP284.
¹³Togawa et al., Journal of Pediatric Gastroenterology and Nutrition 2018, vol. 67, Supp. Supplement 1, pp. S363. Abstract Number: 615.
¹⁴Miloh et al., Gastroenterology 2006, vol. 130, No. 4, Suppl. 2, pp. A759-A760. Meeting Info.: Digestive Disease Week Meeting/107th Annual Meeting of the American-Gastroenterological-Association. Los Angeles, CA, USA. May 19.
¹⁵Dröge et al., Zeitschrift fur Gastroenterologie 2015, vol. 53, No. 12. Abstract Number: A3-27. Meeting Info: 32. Jahrestagung der Deutschen Arbeitsgemeinschaft zum Studium der Leber. Dusseldorf, Germany. 22 Jan 2016-23 Jan 2016
¹⁶Mizuochi et al., Clin Chim Acta. 2012, vol. 413(15-16), p. 1301-1304.
¹⁷Liu et al., Hepatology International 2009, vol. 3, No. 1, p. 184-185. Abstract Number: PE405. Meeting Info: 19th Conference of the Asian Pacific Association for the Study of the Liver. Hong Kong, China. 13 Feb. 2009-16 Feb. 2009
¹⁸McKay et al., Version 2. F1000Res. 2013; 2:32. DOI:10.12688/f1000research.2-32.v2
¹⁹Hasegawa et al., Orphanet J Rare Dis. 2014, vol. 9:89.
²⁰Stone et al., J Biol Chem. 2012, vol. 287(49), p. 41139-51.
²¹Kang et al., J Pathol Transl Med. 2019 May 16. doi:10.4132/jptm.2019.05.03. [Epub ahead of print]
²²Sharma et al., BMC Gastroenterol. 2018, vol. 18(1), p. 107.
²³Uegaki et al., Intern Med. 2008, vol. 47(7), p. 599-602.
²⁴Goldschmidt et al., Hepatol Res. 2016, vol. 46(4), p. 306-311.
²⁵Liu et al., J Pediatr Gastroenterol Nutr. 2010, vol. 50(2), p. 179-183.
²⁶Jung et al., J Pediatr Gastroenterol Nutr. 2007, vol. 44(4), p. 453-458.
²⁷Bounford. University of Birmingham. Dissertation Abstracts International, (2016) Vol. 75, No. 1C. Order No.: AAI10588329. ProQuest Dissertations & Theses.
²⁸Stolz et al., Aliment Pharmacol Ther. 2019, vol. 49(9), p. 1195-1204.
²⁹Ivashkin et al., Hepatology International 2016, vol. 10, No. 1, Supp. SUPPL. 1, pp. S461. Abstract Number: LBO-38. Meeting Info: 25th Annual Conference of the Asian Pacific Association for the Study of the Liver, APASL 2016. Tokyo, Japan. 20 Feb. 2016-24 Feb. 2016
³⁰Blackmore et al., J Clin Exp Hepatol. 2013, vol. 3(2), p. 159-161.
³¹Matte et al., J Pediatr Gastroenterol Nutr. 2010, vol. 51(4), p. 488-493.
³²Squires et al., J Pediatr Gastroenterol Nutr. 2017, vol. 64(3), p. 425-430.
³³Hayshi et al., EBioMedicine. 2018, vol. 27, p. 187-199.
³⁴Nagasaka et al., J Pediatr Gastroenterol Nutr. 2007, vol. 45(1), p. 96-105.
³⁵Wang et al., PLoS One. 2016; vol. 11(4):e0153114.
³⁶Narchi et al., Saudi J Gastroenterol. 2017, vol. 23(5), p. 303-305.
³⁷Alashkar et al., Blood 2015, vol. 126, No. 23. Meeting Info.: 57th Annual Meeting of the American-Society-of-Hematology. Orlando, FL, USA. Dec. 5-8, 2015. Amer Soc Hematol.
³⁸Ferreira et al., Pediatric Transplantation 2013, vol. 17, Supp. SUPPL. 1, pp. 99. Abstract Number: 239. Meeting Info: IPTA 7th Congress on Pediatric Transplantation. Warsaw, Poland. 13 Jul. 2013-16 Jul. 2013.
³⁹Pauli-Magnus et al., J Hepatol. 2005, vol. 43(2), p. 342-357.
⁴⁰Jericho et al., Journal of Pediatric Gastroenterology and Nutrition 2015, vol. 60(3), p. 368-374.
⁴¹van der Woerd et al., PLoS One. 2013, vol. 8(11):e80553.
⁴²Copeland et al., J Gastroenterol Hepatol. 2013, vol. 28(3), p. 560-564.
⁴³Dröge et al., J Hepatol. 2017, vol. 67(6), p. 1253-1264.
⁴⁴Chen et al., Journal of Pediatrics 2002, vol. 140(1), p. 119-124.
⁴⁵Jirsa et al., Hepatol Res. 2004, vol. 30(1), p. 1-3.
⁴⁶van der Woerd et al., Hepatology 2015, vol. 61(4), p. 1382-1391.

In some embodiments, the mutation in ATP8B1 is selected from L127P, G308V, T456M, D554N, F529del, I661T, E665X, R930X, R952X, R1014X, and G1040R.

TABLE 3
Exemplary ABCB11 Mutations

Amino acid position 1 (e.g., M1V)⁹
Amino acid position 4 (e.g., S4X)^{A,64}
Amino acid position 8 (e.g., R8X)⁸⁸
Amino acid position 19 (e.g., G19R)⁵⁶
Amino acid position 24 (e.g., K24X)³⁵
Amino acid position 25 (e.g., S25X)^{5,14}
Amino acid position 26 (e.g., Y26Ifs*7)³⁸
Amino acid position 36 (e.g., D36D)²⁷
Amino acid position 38 (e.g., K38Rfs*24)⁷³
Amino acid position 43 (e.g., V43I)⁵⁷
Amino acid position 49 (e.g., Q49X)⁷³
Amino acid position 50 (e.g., L50S, L50W)⁵⁷
Amino acid position 52 (e.g., R52W²⁶, R52R²⁸)
Amino acid position 56 (e.g., S56L)⁵⁸
Amino acid position 58 (e.g., D58N)⁶²
Amino acid position 62 (e.g., M62K)⁹
Amino acid position 66 (e.g., S66N)¹⁷
Amino acid position 68 (e.g., C68Y)⁴¹
Amino acid position 50 (e.g., L50S)^{5,7}
Amino acid position 71 (e.g., L71H)⁷³
Amino acid position 74 (e.g., I74R)⁷¹
Amino acid position 77 (e.g., P77A)⁷³
Amino acid position 87 (e.g., T87R)⁶⁷
Amino acid position 90 (e.g., F90F)^{7,27}
Amino acid position 93 (e.g., Y93S¹³, Y93X⁸⁸)
Amino acid position 96 (e.g., E96X)⁸⁸
Amino acid position 97 (e.g., L97X)³⁹
Amino acid position 101 (e.g., Q101Dfs*8)⁹
Amino acid position 107 (e.g., C107R)³⁶
Amino acid position 112 (e.g., I112T)⁹
Amino acid position 114 (e.g., W114R)^{2,9}
Amino acid position 123 (e.g. M123T)⁶⁷
Amino acid position 127 (e.g., T127Hfs*6)⁵
Amino acid position 129 (e.g., C129Y)²⁵
Amino acid position 130 (e.g., G130G)⁷⁷
Amino acid position 134 (e.g., I134I)²⁸
Amino acid position 135 (e.g., E135K^{7,13}, E135L¹⁷)
Amino acid position 137 (e.g., E137K)⁷
Amino acid position 157 (e.g., Y157C)⁵
Amino acid position 161 (e.g., C161X)³⁹
Amino acid position 164 (e.g., V164Gfs*7³⁰, V164I⁸⁵)
Amino acid position 167 (e.g., A167S⁴, A167V⁷, A167T^{9,17})
Amino acid position 181 (e.g., R181I)³⁵
Amino acid position 182 (e.g., I182K)⁹
Amino acid position 183 (e.g., M183V⁸, M183T⁹)
Amino acid position 185 (e.g., M185I)⁷³
Amino acid position 186 (e.g., E186G)^{2,7,22}
Amino acid position 188 (e.g., G188W)⁷³
Amino acid position 194 (e.g., S194P)⁷
Amino acid position 198 (e.g., L198P)⁷
Amino acid position 199 (e.g., N199Ifs*15X)⁸⁸
Amino acid position 206 (e.g., I206V)²⁸
Amino acid position 212 (e.g., A212T)⁷³
Amino acid position 217 (e.g., M217R)⁸⁸
Amino acid position 225 (e.g., T225P)⁵⁷
Amino acid position 226 (e.g., S226L)⁹
Amino acid position 232 (e.g., L232Cfs*9)⁹
Amino acid position 233 (e.g., L233S)⁸⁶
Amino acid position 238 (e.g., G238V)^{2,7}
Amino acid position 242 (e.g., T242I)^{5,7}
Amino acid position 245 (e.g., I245Tfs*26)⁵⁷
Amino acid position 256 (e.g., A256G)⁹
Amino acid position 260 (e.g., G260D)⁷
Amino acid position 269 (e.g., Y269Y)²⁷
Amino acid position 277 (e.g., A277E)⁷⁷
Amino acid position 283 (e.g., E283D)⁷³
Amino acid positions 212 and 283 (e.g., A212T+E283D)⁷³
Amino acid position 284 (e.g., V284L^{7,39}, V284A⁷, V284D²³)
Amino acid position 297 (e.g., E297G^{1,2,5,7}, E297K⁷)
Amino acid position 299 (e.g., R299K)²⁸
Amino acid position 303 (e.g., R303K⁸, R303M⁶³ R303fsX321⁸³)
Amino acid position 304 (e.g., Y304X)²⁶
Amino acid position 312 (e.g., Q312H)⁷
Amino acid position 313 (e.g., R313S)^{5,7}
Amino acid position 314 (e.g., W314X)⁵⁷
Amino acid position 318 (e.g., K318Rfs*26)²⁹
Amino acid position 319 (e.g., G319G)⁷
Amino acid position 327 (e.g., G327E)^{5,7}
Amino acid position 330 (e.g., W330X)²⁴
Amino acid position 336 (e.g., C336S)^{2,7}
Amino acid position 337 (e.g., Y337H)^{21,27}
Amino acid position 342 (e.g., W342G)⁵⁰
Amino acid position 354 (e.g., R354X)⁹
Amino acid position 361 (e.g., Q361X⁵⁷, Q361R⁷⁴)
Amino acid position 366 (e.g., V366V²⁸, V366D⁵⁷)
Amino acid position 368 (e.g., V368Rfs*27)⁵
Amino acid position 374 (e.g., G374S)³
Amino acid position 380 (e.g., L380Wfs*18)⁵
Amino acid position 382 (e.g., A382G)⁸⁸
Δ Amino acid positions 382-388⁵
Δ Amino acid positions 383-389⁵⁷
Amino acid position 387 (e.g., R387H)⁹
Amino acid position 390 (e.g., A390P)^{5,7}
Amino acid position 395 (e.g., E395E)²⁸
Amino acid position 404 (e.g., D404G)⁹
Amino acid position 410 (e.g., G410D)^{5,7}
Amino acid position 413 (e.g., L413W)^{5,7}
Amino acid position 415 (e.g., R415X)⁴²
Amino acid position 416 (e.g., I416I)²⁷
Amino acid position 420 (e.g., I420T)⁹
Amino acid position 423 (e.g., H423R)¹³
Amino acid position 432 (e.g., R432T)^{1,2,7}
Amino acid position 436 (e.g., K436N)⁴⁰
Amino acid position 440 (e.g., D440E)⁸⁸
Amino acid position 444 (e.g., V444A)²
Amino acid position 454 (e.g., V454X)⁴⁹
Amino acid position 455 (e.g., G455E)⁹
Amino acid position 457 (e.g., S457Vfs*23)⁸⁸
Amino acid position 461 (e.g., K461E)^{2,7}
Amino acid position 462 (e.g., S462R)⁸⁸
Amino acid position 463 (e.g., T463I)^{5,7}
Amino acid position 466 (e.g., Q466K)^{5,7}
Amino acid position 470 (e.g., R470Q^{5,7}, R470X⁹)
Amino acid position 471 (e.g., Y472X)⁵
Amino acid position 472 (e.g., Y472C^{5,27}, Y472X¹⁴)
Amino acid position 473 (e.g., D473Q³⁵, D473V⁸⁸)
Amino acid position 475 (e.g., C475X)²⁹
Amino acid position 481 (e.g., V481E)^{5,7}
Amino acid position 482 (e.g., D482G)^{2,5,7}
Amino acid position 484 (e.g., H484Rfs*5)⁹
Amino acid position 487 (e.g., R487H², R487P⁵)
Amino acid position 490 (e.g., N490D)^{5,7}
Amino acid position 493 (e.g., W493X)⁸
Amino acid positon 496 (e.g., D496V)⁸⁸
Amino acid position 498 (e.g., I498T)^{2,7}
Amino acid position 499 (e.g., G499E)⁷³
Amino acid position 501 (e.g., V501G)⁶⁸
Amino acid position 504 (e.g., E504K)⁷⁹
Amino acid position 510 (e.g., T510T)⁷
Amino acid position 512 (e.g., I512T)^{5,7}
Amino acid position 515 (e.g., N515T^{5,7}, N515D⁶⁴)
Amino acid position 516 (e.g., I516M)¹⁷
Amino acid position 517 (e.g., R517H)^{5,7}
Amino acid position 520 (e.g., R520X)⁵
Amino acid position 523 (e.g., A523G)¹³
Amino acid position 528 (e.g., I528Sfs*21⁵, I528X⁹, I528T⁷³)
Amino acid position 535 (e.g., A535A⁷, A535X⁸⁹)
Amino acid position 540 (e.g., F540L)⁴⁶
Amino acid position 541 (e.g., I541L^{5,7}, I541T^{5,17})
Amino acid position 546 (e.g., Q546K³⁹, Q546H⁷³)
Amino acid position 548 (e.g., F548Y)^{5,7}
Amino acid position 549 (e.g., D549V)⁹
Amino acid position 554 (e.g., E554K)²¹
Amino acid position 556 (e.g., G556R)⁶⁷
Amino acid position 558 (e.g., Q558H)²³
Amino acid position 559 (e.g., M559T)⁵⁷
Amino acid position 562 (e.g., G562D^{5,7}, G562S⁷³)
Amino acid position 570 (e.g., A570T^{2,5,7}, A570V²⁶)
Amino acid position 575 (e.g., R575X^{2,5}, R575Q²¹)
Amino acid position 580 (e.g., L580P)⁵⁷
Amino acid position 586 (e.g., T586I)⁷
Amino acid position 587 (e.g., S587X)⁷³
Amino acid position 588 (e.g., A588V^{5,7}, A588P⁷³)
Amino acid position 591 (e.g., N591S)^{2,7}
Amino acid position 593 (e.g., S593R)^{2,7}
Amino acid position 597 (e.g., V597V⁹, V597L¹³)
Amino acid position 603 (e.g., K603K)⁵⁵
Amino acid position 609 (e.g., H609Hfs*46)²⁶
Amino acid position 610 (e.g., I610Gfs*45⁹, I610T⁵⁷)⁹
Amino acid position 615 (e.g., H615R)²⁶
Amino acid position 616 (e.g., R616G²⁸, R616H⁷³)
Amino acid position 619 (e.g., T619A)²⁸
Amino acid position 623 (e.g., A623A)²⁸
Amino acid position 625 (e.g., T625Nfs*5)²⁶
Amino acid position 627 (e.g., I627T)⁷
Amino acid position 628 (e.g., G628Wfs*3)⁷⁰
Amino acid position 636 (e.g., E636G)²
Amino acid position 648 (e.g., G648Vfs*6⁵, G648V⁵⁰)
Amino acid position 655 (e.g., T655I)⁷
Amino acid position 669 (e.g., I669V)²⁶
Amino acid position 676 (e.g., D676Y)¹¹
Amino acid position 677 (e.g., M677V)^{7,13}
Amino acid position 679 (e.g., A679V)⁵⁸
Amino acid position 685 (e.g., G685W)⁶⁰
Amino acid position 696 (e.g., R696W²⁷, R696Q⁵⁸)
Amino acid position 698 (e.g., R698H^{7,9}, R698K⁶¹, R698C⁸⁸)
Amino acid position 699 (e.g., S699P)⁹
Amino acid position 701 (e.g., S701P)⁵⁸
Amino acid position 702 (e.g., Q702X)⁸⁹
Amino acid position 709 (e.g., E709K)⁷
Amino acid position 710 (e.g., P710P)⁷
Amino acid position 712 (e.g., L712L)²⁸
Amino acid position 721 (e.g., Y721C)⁸⁸
Amino acid position 729 (e.g., D724N)³⁹
Amino acid position 731 (e.g., P731S)²³
Amino acid position 740 (e.g., P740Qfs*6)⁷³
Amino acid position 758 (e.g., G758R)⁵
Amino acid position 766 (e.g., G766R)^{5,24}
Amino acid position 772 (e.g., Y772X)⁵
Amino acid position 804 (e.g., A804A)⁷
Amino acid position 806 (e.g., G806D⁴⁴, G806G⁵⁵)
Amino acid position 809 (e.g., S809F)⁸¹
Amino acid position 817 (e.g., G817G)⁸⁸
Amino acid position 818 (e.g., Y818F)⁷
Amino acid position 824 (e.g., G824E)⁴²
Amino acid position 825 (e.g., G825G)⁷³
Amino acid position 830 (e.g., R830Gfs*28)⁷³
Amino acid position 832 (e.g., R832C^{7,26}, R832H⁴¹)
Amino acid position 842 (e.g., D842G)²
Amino acid position 848 (e.g., D848N)⁷³
Amino acid position 855 (e.g., G855R)¹¹
Amino acid position 859 (e.g., T859R)^{5,7}
Amino acid position 865 (e.g., A865V)²⁷
Amino acid position 866 (e.g., S866A)⁵⁷
Amino acid position 868 (e.g., V868D)⁷³
Amino acid position 869 (e.g., Q869P)⁷³
Amino acid position 875 (e.g., Q875X)⁷³
Amino acid position 877 (e.g., G877R)⁵⁶
Amino acid position 879 (e.g., I879R)⁸⁸
Amino acid position 893 (e.g., A893V)⁵⁷
Amino acid position 901 (e.g., S901R¹⁷, S901I⁷³)
Amino acid position 903 (e.g., V903G)⁵⁷
Δ Amino acid position 919¹²
Amino acid position 923 (e.g., T923P)^{2,7}
Amino acid position 926 (e.g., A926P)^{2,7}
Amino acid position 928 (e.g., R928X¹⁵, R928Q⁴⁰)
Amino acid position 930 (e.g., K930X⁵, K930Efs*79^{5,10}, K930Efs*49²⁶)
Amino acid position 931 (e.g., Q931P)²⁷
Amino acid position 945 (e.g., S945N)⁵⁷
Amino acid position 948 (e.g., R948C)^{5,7,26}
Amino acid position 958 (e.g., R958Q)²⁸
Amino acid position 969 (e.g., K969K)⁸⁸
Δ Amino acid positions 969-972⁵
Amino acid position 973 (e.g., T973I)⁵⁷
Amino acid position 976 (e.g., Q976R⁵⁸, Q976X⁸⁸)
Amino acid position 979 (e.g., N979D)^{5,7}
Amino acid position 981 (e.g., Y981Y)²⁸
Amino acid position 982 (e.g., G982R)^{2,5,7}
Amino acid positions 444 and 982 (e.g., V444A+G982R)³⁸
Amino acid position 995 (e.g., A995A)²⁸
Amino acid position 1001 (e.g., R1001R)⁹
Amino acid position 1003 (e.g., G1003R)²⁴
Amino acid position 1004 (e.g., G1004D)^{2,7}
Amino acid position 1027 (e.g., S1027R)²⁶
Amino acid position 1028 (e.g., A1028A^{7,10,88}, A1028E⁸⁸)
Amino acid position 1029 (e.g., T1029K)⁵
Amino acid position 1032 (e.g., G1032R)¹²
Amino acid position 1041 (e.g., Y1041X)⁹
Amino acid position 1044 (e.g., A1044P)⁸⁸
Amino acid position 1050 (e.g., R1050C)^{2,7,57}
Amino acid position 1053 (e.g., Q1053X)⁵⁷
Amino acid position 1055 (e.g., L1055P)³⁶
Amino acid position 1057 (e.g., R1057X², R1057Q⁵⁸)
Amino acid position 1058 (e.g., Q1058Hfs*38⁹, Q1058fs*38¹⁷,
Q1058X⁷³)
Amino acid position 1061 (e.g., I1061Vfs*34)⁹
Amino acid position 1083 (e.g., C1083Y)⁴⁷
Amino acid position 1086 (e.g., T1086T)²⁸
Amino acid position 1090 (e.g., R1090X)^{2,5}
Amino acid position 1099 (e.g., L1099Lfs*38)²⁶
Amino acid position 1100 (e.g., S1100Qfs*38)¹³
Amino acid position 1110 (e.g., A1110E)^{5,7}
Amino acid position 1112 (e.g., V1112F)⁷⁰
Amino acid position 1116 (e.g., G1116R⁷, G1116F^{9,17}, G1116E³⁶)
Amino acid position 1120 (e.g., S1120N)⁸⁸
Amino acid position 1128 (e.g., R1128H^{2,7}, R1128C^{5,7,13})
Amino acid position 1131 (e.g., D1131V)²⁷
Amino acid position 1144 (e.g., S1144R)⁷
Amino acid position 1147 (e.g., V1147X)⁵
Amino acid position 1153 (e.g., R1153C^{2,5,7}, R1153H⁵)
Amino acid position 1154 (e.g., S1154P)^{5,7}
Amino acid position 1162 (e.g., E1162X)³⁹
Δ Amino acid position 1165⁸⁸
Amino acid position 1164 (e.g., V1164Gfs*7)
Amino acid position 1173 (e.g., N1173D)⁵⁷
Amino acid position 1175 (e.g., K1175T)⁵⁸
Amino acid position 1186 (e.g., E1186K)⁷
Amino acid position 1192 (e.g., A1192Efs*50)⁹
Amino acid position 1196 (e.g., Q1196X)⁸⁸
Amino acid position 1197 (e.g., L1197G)⁷
Amino acid position 1198 (e.g., H1198R)²⁷
Amino acid position 1204 (e.g., L1204P)⁸⁸
Amino acid position 1208 (e.g. Y1208C)⁷³
Amino acid position 1210 (e.g., T1210P^{5,7}, T1210F⁵⁷)
Amino acid position 1211 (e.g., N1211D)⁷
Amino acid position 1212 (e.g., V1212F)³⁶
Amino acid position 1215 (e.g., Q1215X)⁵
Amino acid position 1221 (e.g., R1221K)⁵³
Amino acid position 1223 (e.g., E1223D)⁷
Amino acid position 1226 (e.g., R1226P)⁷³
Amino acid position 1228 (e.g., A1228V)⁷
Amino acid position 1231 (e.g., R1231W^{5,7}, R1231Q^{5,7})
Amino acid position 1232 (e.g., A1232D)¹⁷
Amino acid position 1235 (e.g., R1235X)^{5,12}
Amino acid position 1242 (e.g., L1242I)^{5,7}
Amino acid position 1243 (e.g., D1243G)⁶⁷
Amino acid position 1249 (e.g., L1249X)⁷³
Amino acid position 1256 (e.g., T1256fs*1296)⁸³
Amino acid position 1268 (e.g., R1268Q)^{2,7}
Amino acid position 1276 (e.g., R1276H)³⁰
Amino acid position 1283 (e.g., A1283A²⁸, A1283V⁸⁸)
Amino acid position 1292 (e.g., G1292V)⁷³
Amino acid position 1298 (e.g., G1298R)⁵
Amino acid position 1302 (e.g., E1302X)⁵
Amino acid position 1311 (e.g., Y1311X)⁵⁷
Amino acid position 1316 (e.g., T1316Lfs*64)¹⁵
Amino acid position 1321 (e.g., S1321N)⁵⁷
Intron 4 ((+3)A>C)¹
IVS4−74A>T⁸⁹
Splice site mutation 3′ Intron 5 c.3901G>A⁵
Splice site mutation 5; Intron 7 c.6111G>A⁵
Splice site mutation IVS7+1G>A¹⁴
IVS7+5G>A⁴⁰
IVS8+1G>C⁷⁶
Splice site mutation 5′ Intron 9 c.9081delG⁵
Splice site mutation 5′ Intron 9 c.9081G>T⁵
Splice site mutation 5′ Intron 9 c.9081G>A⁵
Splice site mutation IVS9+1G>T¹⁴
Splice site mutation 3′ Intron 13 c.143513_1435−8del⁵
Splice site mutation IVS13del−13\hat{ }−8¹⁴
Splice site mutation 3′ Intron 16 c.20128T>G⁵
Splice site mutation IVS16−8T>G¹⁴
Splice site mutation 5′ Intron 18 c.21781G>T⁵
Splice site mutation 5′ Intron 18 c.21781G>A⁵
Splice site mutation 5′ Intron 18 c.21781G>C⁵
Splice site mutation 3′ Intron 18 c.21792A>G⁵
Splice site mutation IVS18+1G>A¹⁴
Splice site mutation 5′ Intron 19 c.2343+1G>T⁵
Splice site mutation 5′ Intron 19 c.2343+2T>C⁵
Splice site mutation IVS19+2T>C¹⁴
Splice site mutation IVS19+1G>A²²
Splice site mutation 3′ Intron 21 c.26112A>T⁵
IVS22+3A>G⁸⁹
IVS 23−8 G-A³⁶
IVS24+5G>A⁵¹
Splice site mutation 5′ Intron 24 c.32131delG⁵
IVS35−6C>G⁸⁹
Putative splice mutation 1198−1G>C¹⁷
Putative splice mutation 1810−3C>G¹⁷
Putative splice mutation 2178+1G>A¹⁷
Putative splice mutation 2344−1G>T¹⁷
Putative splice mutation c.2611−2A>T³⁹
Putative splice mutation 3213+1_3213+2delinsA¹⁷
c.−24C>A^{44,78}
c.76 13 G>T⁹
c.77−19T>A⁵²
c.90_93delGAAA¹⁸
c.124G>A⁶⁹
c.150+3 A>C¹⁰
174C>T⁵⁴
c.245T>C⁸⁷
c.249_250insT¹⁸
270T>C⁵⁴
402C>T⁵⁴
585G>C⁵⁴
c.611+1G>A⁷⁰
c.611+4A>G³⁶
c.612−15_−6del10bp⁵⁵
c.625A>C³¹
c.627+5G>T³¹
c.625A>C/c.627+5G>T³¹
696G>T⁵⁴
c. 784+1G>C⁴⁹
807T>C⁵⁴
c.886C>T³¹
c.890A>G⁵⁹
c.908+1G>A⁵⁷
c.908+5G>A⁵⁵
c.908delG⁵⁹
c.909−15A>G⁶⁶
957A>G⁵⁴
c.1084−2A>G⁵⁷
1145 1 bp deletion⁹⁰
1281C>T^{54,57}
c.1309−165C>T¹⁹
c.1434+174G>A¹⁹
c.1434+70C>T¹⁹
c.1530C>A⁵⁷
c.1587-1589delCTT³¹
c.1621A>C^{33,59}
c.1638+32T>C⁶⁶
c.1638+80C>T⁶⁶
1671C>T⁵⁴
1791G>T⁵⁴
1939delA¹⁴
c.2075+3A>G⁵³
c.2081T>A³¹
c.2093G>A⁶⁵
2098delA¹⁶
c.2138−8T>G⁶⁷
2142A>G⁵⁴
c.2178+1G>T^{36,39}
c.2179−17C>A⁶⁶
c.2344−157T>G⁶⁶
c.2344−17T>C⁶⁶
c.2417G>A⁷⁸
c.2541delG⁸⁷
c.2620C>T^{32,33}
c.2815−8A>G⁵⁵
c.3003A>G³⁷
c.3084A>G^{48,54}
c.3213+4 A>G^{9,37}
c.3213+5 G>A⁹
c.3268C>T⁷⁵
3285A>G⁵⁴
c.3382C>T⁷⁵
3435A>G⁵⁴
c.3491delT⁷²
c.3589C>T⁵⁷
c.3765(+1+5)del5⁴²
c.3766−34A>G⁶⁶
c.3767−3768insC⁶
c.3770delA⁶⁷
c.3826C>T⁷²
c.3846C>T⁵⁷
c.3929delG⁶⁷
c.*236A>G⁶⁶
1145delC⁸
Ex13_Ex17del⁸²

TABLE 4
Selected ABCB11 Mutations Associated with PFIC-2

Amino acid position 1 (e.g., M1V)⁹
Amino acid position 4 (e.g., S4X)⁶⁴
Amino acid position 19 (e.g., G19R)⁵⁶
Amino acid position 25 (e.g., S25X)¹⁴
Amino acid position 26 (e.g., Y26Ifs*7)³⁸
Amino acid position 50 (e.g., L50S)^{7,57}
Amino acid position 52 (e.g., R52W)²⁶
Amino acid position 58 (e.g., D58N)⁶²
Amino acid position 62 (e.g., M62K)⁹
Amino acid position 66 (e.g., S66N)¹⁷
Amino acid position 68 (e.g., C68Y)⁴¹
Amino acid position 93 (e.g., Y93S)¹³
Amino acid position 101 (e.g., Q101Dfs*8)⁹
Amino acid position 107 (e.g., C107R)³⁶
Amino acid position 112 (e.g., I112T)⁹
Amino acid position 114 (e.g., W114R)^{2,9}
Amino acid position 129 (e.g., C129Y)²⁵
Amino acid position 135 (e.g., E135K¹³, E135L¹⁷)
Amino acid position 167 (e.g., A167V⁷, A167T^{9,17})
Amino acid position 182 (e.g., I182K)⁹
Amino acid position 183 (e.g., M183V⁸, M183T⁹)
Amino acid position 225 (e.g., T225P)⁵⁷
Amino acid position 226 (e.g., S226L)⁹
Amino acid position 232 (e.g., L232Cfs*9)⁹
Amino acid position 233 (e.g., L233S)⁸⁶
Amino acid position 238 (e.g., G238V)^{2,7}
Amino acid position 242 (e.g., T242I)⁷
Amino acid position 245 (e.g., I245Tfs*26)⁵⁷
Amino acid position 256 (e.g., A256G)⁹
Amino acid position 260 (e.g., G260D)⁵⁷
Amino acid position 284 (e.g., V284L)⁷
Amino acid position 297 (e.g., E297G)^{2,7}
Amino acid position 303 (e.g., R303K⁸, R303M⁶³, R303fsX321⁸³)
Amino acid position 304 (e.g., Y304X)²⁶
Amino acid position 312 (e.g., Q312H)⁷
Amino acid position 313 (e.g., R313S)⁷
Amino acid position 314 (e.g., W314X)⁵⁷
Amino acid position 318 (e.g., K318Rfs*26)²⁹
Amino acid position 327 (e.g., G327E)⁷
Amino acid position 330 (e.g., V330X)²⁴
Amino acid position 336 (e.g., C336S)^{2,7}
Amino acid position 337 (e.g., Y337H)²¹
Amino acid position 342 (e.g., W342G)⁵⁰
Amino acid position 354 (e.g., R354X)⁹
Amino acid position 361 (e.g., Q361X)⁵⁷
Amino acid position 366 (e.g., V366D)⁵⁷
Amino acid position 386 (e.g., G386X)³⁴
Δ Amino acid positions 383-389⁵⁷
Amino acid position 387 (e.g., R387H)⁹
Amino acid position 390 (e.g., A390P)⁷
Amino acid position 410 (e.g., G410D)⁷
Amino acid position 413 (e.g., L413W)⁷
Amino acid position 415 (e.g., R415X)⁴²
Amino acid position 420 (e.g., I420T)⁹
Amino acid position 454 (e.g., V454X)⁴⁹
Amino acid position 455 (e.g., G455E)⁹
Amino acid position 461 (e.g., K461E)^{2,7}
Amino acid position 463 (e.g., T463I)⁷
Amino acid position 466 (e.g., Q466K)⁷
Amino acid position 470 (e.g., R470Q⁷, R470X⁹)
Amino acid position 472 (e.g., Y472X¹⁴, Y472C²⁷)
Amino acid position 475 (e.g., C475X)²⁹
Amino acid position 481 (e.g., V481E)⁷
Amino acid position 482 (e.g., D482G)^{2,7}
Amino acid position 484 (e.g., H484Rfs*5)⁹
Amino acid position 487 (e.g., R487H², R487P⁸⁴)
Amino acid position 490 (e.g., N490D)⁷
Amino acid position 493 (e.g., W493X)⁸
Amino acid position 498 (e.g., I498T)⁷
Amino acid position 501 (e.g., V501G)⁶⁸
Amino acid position 512 (e.g., I512T)⁷
Amino acid position 515 (e.g., N515T⁷, N515D⁶⁴)
Amino acid position 516 (e.g., I516M)¹⁷
Amino acid position 517 (e.g., R517H)⁷
Amino acid position 520 (e.g., R520X)⁵⁷
Amino acid position 523 (e.g., A523G)¹³
Amino acid position 528 (e.g., I528X)⁹
Amino acid position 540 (e.g., F540L)⁴⁶
Amino acid position 541 (e.g., I541L⁷, I541T¹⁷)
Amino acid position 548 (e.g., F548Y)⁷
Amino acid position 549 (e.g., D549V)⁹
Amino acid position 554 (e.g., E554K)²¹
Amino acid position 559 (e.g., M559T)⁵⁷
Amino acid position 562 (e.g., G562D)⁷
Amino acid position 570 (e.g., A570T⁷, A570V²⁶)
Amino acid position 575 (e.g., R575X², R575Q²¹)
Amino acid position 588 (e.g., A588V)⁷
Amino acid position 591 (e.g., N591S)^{9,17}
Amino acid position 593 (e.g., S593R)^{2,7}
Amino acid position 597 (e.g., V597V⁹, V597L¹³)
Amino acid positions 591 and 597 (e.g., N591S + V597V)⁹
Amino acid position 603 (e.g., K603K)⁵⁵
Amino acid position 609 (e.g., H609Hfs*46)²⁶
Amino acid position 610 (e.g., I610Gfs*45)⁹
Amino acid position 615 (e.g., H615R)²⁶
Amino acid position 625 (e.g., T625Nfs*5)²⁶
Amino acid position 627 (e.g., I627T)⁷
Amino acid position 636 (e.g., E636G)²
Amino acid position 669 (e.g., I669V)²⁶
Amino acid position 698 (e.g., R609H)⁹
Amino acid positions 112 and 698 (e.g., I112T + R698H)⁹
Amino acid position 699 (e.g., S699P)⁹
Amino acid position 766 (e.g., G766R)²⁴
Amino acid position 806 (e.g., G806G)⁵⁵
Amino acid position 824 (e.g., G824E)⁴²
Amino acid position 832 (e.g., R832C^{7,26}, R832H⁴¹)
Amino acid position 842 (e.g., D842G)²
Amino acid position 859 (e.g., T859R)⁷
Amino acid position 865 (e.g., A865V)⁴⁵
Amino acid position 877 (e.g., G877R)⁵⁶
Amino acid position 893 (e.g., A893V)⁵⁷
Amino acid position 901 (e.g., S901R)¹⁷
Amino acid position 903 (e.g., V903G)⁵⁷
Δ Amino acid position 919¹²
Amino acid position 928 (e.g., R928X)^{15,21}
Amino acid position 930 (e.g., K930Efs*79¹⁰, K930Efs*49²⁶)
Amino acid position 948 (e.g., R948C)^{7,26}
Amino acid position 979 (e.g., N979D)⁷
Amino acid position 982 (e.g., G982R)^{2,7}
Amino acid positions 444 and 982 (e.g., V444A + G982R)³⁸
Amino acid position 1001 (e.g., R1001R)⁹
Amino acid position 1003 (e.g., G1003R)²⁴
Amino acid position 1004 (e.g., G1004D)^{2,7}
Amino acid position 1027 (e.g., S1027R)²⁶
Amino acid position 1028 (e.g., A1028A)¹⁰
Amino acid position 1032 (e.g., G1032R)¹²
Amino acid position 1041 (e.g., Y1041X)⁹
Amino acid position 1050 (e.g., R1050C)⁵⁷
Amino acid position 1053 (e.g., Q1053X)⁵⁷
Amino acid position 1055 (e.g., L1055P)³⁶
Amino acid position 1057 (e.g., R1057X)²
Amino acid position 1058 (e.g., Q1058Hfs*38⁹, Q1058fs*38¹⁷)
Amino acid position 1061 (e.g., I1061Vfs*34)⁹
Amino acid position 1083 (e.g., C1083Y)⁴⁷
Amino acid position 1090 (e.g., R1090X)²
Amino acid position 1099 (e.g., L1099Lfs*38)²⁶
Amino acid position 1100 (e.g., S1100Qfs*38)¹³
Amino acid position 1110 (e.g., A1110E)⁷
Amino acid position 1116 (e.g., G1116R⁷, G1116F^{9,17}, G1116E³⁶)
Amino acid position 1128 (e.g., R1128C)^{7,13}
Amino acid position 1131 (e.g., D1131V)²⁷
Amino acid position 1144 (e.g., S1144R)⁷
Amino acid position 1153 (e.g., R1153C^{2,7}, R1153H^{7,26})
Amino acid position 1154 (e.g., S1154P)⁷
Amino acid position 1173 (e.g., N1173D)⁵⁷
Amino acid position 1192 (e.g., A1192Efs*50)⁹
Amino acid position 1198 (e.g., H1198R)²⁷
Amino acid position 1210 (e.g., T1210P⁷, T1210F⁵⁷)
Amino acid position 1211 (e.g., N1211D)⁷
Amino acid position 1212 (e.g., V1212F)³⁶
Amino acid position 1231 (e.g., R1231W⁷, R1223Q⁷)
Amino acid position 1232 (e.g., A1232D)¹⁷
Amino acid position 1235 (e.g., R1235X)¹²
Amino acid position 1242 (e.g., L1242I)⁷
Amino acid position 1256 (e.g., T1256fs*1296)⁸³
Amino acid position 1268 (e.g., R1268Q)^{2,7}
Amino acid position 1302 (e.g. E1302X)⁵⁷
Amino acid position 1311 (e.g., Y1311X)⁵⁷
Amino acid position 1316 (e.g., T1316Lfs*64)¹⁵
Intron 4 ((+3)A > C)¹
Splice site mutation IVS7 + 1G > A¹⁴
IVS8 + 1G > C⁷⁶
Splice site mutation IVS9 + 1G > T¹⁴
Splice site mutation IVS13del-13\hat{ }-8¹⁴
Splice site mutation IVS16 − 8T > G¹⁴
Splice site mutation IVS18 + 1G > A¹⁴
Splice site mutation IVS19 + 2T > C¹⁴
IVS 23 − 8 G − A³⁶
IVS24 + 5G > A⁵¹
Putative splice mutation 1198 − 1G > C¹⁷
Putative splice mutation 1810 − 3C > G¹⁷
Putative splice mutation 2178 + 1G > A¹⁷
Putative splice mutation 2344 − 1G > T¹⁷
Putative splice mutation 3213 + 1_3213 + 2delinsA¹⁷
c.-24C > A⁷⁸
c.76 13 G > T⁹
c.77 − 19T > A⁵²
c.90_93delGAAA¹⁸
c.124G > A⁶⁹
c.150 + 3 A > C¹⁰
c.249_250insT¹⁸
c.611 + 1G > A⁸⁴
c.611 + 4A > G³⁶
c.612 − 15_-6del10bp⁵⁵
c.625A > C³¹
c.627 + 5G > T³¹
c.625A > C/c.627 + 5G > T³¹
c.886C > T³¹
c.890A > G⁵⁹
c.908 + 1G > A⁵⁷
c.908 + 5G > A⁵⁵
c.908delG⁵⁹
1273 1 bp deletion⁹¹
c.1084 − 2A > G⁵⁷
c.1445A > G⁵⁹
c.1587-1589delCTT³¹
c.1621A > C⁵⁹
1939delA¹⁴
c.2081T > A³¹
2098delA¹⁶
c.2343 + 1 G > T⁸⁰
c.2178 + 1G > T³⁶
c.2417G > A⁷⁸
c.2620C > T³²
c.2815 − 8A > G⁵⁵
c.3003A > G³⁷
c.3213 + 4 A > G^{9,37}
c.3213 + 5 G > A⁹
c.3268C > T⁷⁵
c.3382C > T⁷⁵
c.3765(+1 + 5)del5⁴²
c.3767-3768insC⁶
1145delC⁸
Ex13_Ex17del⁸²
^{A} A mutation to ′X′ denotes an early stop codon

### References for Tables 3 and 4

¹Noe et al., J Hepatol. 2005, vol. 43(3), p. 536-543.
²Lam et al., Am J Physiol Cell Physiol. 2007, vol. 293(5), p. C1709-16.
³Stindt et al., Liver Int. 2013, vol. 33(10), p. 1527-1735.
⁴Gao et al., Shandong Yiyao 2012, vol. 52(10), p. 14-16.
⁵Strautnieks et al., Gastroenterology. 2008, vol. 134(4), p. 1203-1214.
⁶Kagawa et al., Am J Physiol Gastrointest Liver Physiol. 2008, vol. 294(1), p. G58-67.
⁷Byrne et al., Hepatology. 2009, vol. 49(2), p. 553-567.
⁸Chen et al., J Pediatr. 2008, vol. 153(6), p. 825-832.
⁹Davit-Spraul et al., Hepatology 2010, vol. 51(5), p. 1645-1655.
¹⁰Dröge et al., Sci Rep. 2016, vol. 6:24827.
¹¹Lang et al., Pharmacogenet Genomics. 2007, vol. 17(1), p. 47-60.
¹²Ellinger et al., World J Gastroenterol. 2017, vol. 23(29), p. :5295-5303.
¹³Vitale et al., J Gastroenterol. 2018, vol. 53(8), p. 945-958.
¹⁴Knisely et al., Hepatology. 2006, vol. 44(2), p. 478-86.
¹⁵Ellis et al., Hepatology. 2018, vol. 67(4), p. 1531-1545.
¹⁶Lam et al., J Hepatol. 2006, vol. 44(1), p. 240-242.
¹⁷Varma et al., Hepatology 2015, vol. 62(1), p. 198-206.
¹⁸Treepongkaruna et al., World J Gastroenterol. 2009, vol. 15(34), p. 4339-4342.
¹⁹Zarenezhad et al., Hepatitis Monthly: 2017, vol. 17(2); e43500.
²⁰Hayashi et al., Hepatol Res. 2016, vol. 46(2), p. 192-200.
²¹Guorui et al., Linchuang Erke Zazhi 2013, vol. 31(10), 905-909.
²²van Mil et al., Gastroenterology. 2004, vol. 127(2), p. 379-384.
²³Anzivino et al., Dig Liver Dis. 2013, vol. 45(3), p. 226-232.
²⁴Park et al., World J Gastroenterol. 2016, vol. 22(20), p. 4901-4907.
²⁵Imagawa et al., J Hum Genet. 2018, vol. 63(5), p. 569-577.
²⁶Giovannoni et al., PLoS One. 2015, vol. 10(12):e0145021.
²⁷Hu et al., Mol Med Rep. 2014, vol. 10(3), p. 1264-1274.
²⁸Lang et al,. Drug Metab Dispos. 2006, vol. 34(9), p. 1582-1599.
²⁹Masahata et al., Transplant Proc. 2016, vol. 48(9), p. 3156-3162.
³⁰Holz et al., Hepatol Commun. 2018, vol. 2(2), p. 152-154.
³¹Li et al., Hepatology International 2017, vol. 11, No. 1, Supp. Supplement 1, pp. S180. Abstract Number: OP284.
³²Francalanci et al., Laboratory Investigation 2011, vol. 91, Supp. SUPPL. 1, pp. 360A. Abstract Number: 1526.
³³Francalanci et al., Digestive and Liver Disease 2010, vol. 42, Supp. SUPPL. 1, pp. S16. Abstract Number: T.N.S.
³⁴Shah et al., J Pediatr Genet. 2017, vol. 6(2), p. 126-127.
³⁵Gao et al., Hepatitis Monthly 2017, vol. 17(10), e55087/1-e55087/6.
³⁶Evason et al., Am J Surg Pathol. 2011, vol. 35(5), p. 687-696.
³⁷Davit-Spraul et al., Mol Genet Metab. 2014, vol. 113(3), p. 225-229.
³⁸Maggiore et al., J Hepatol. 2010, vol. 53(5), p. 981-6.
³⁹McKay et al., Version 2. F1000Res. 2013; 2:32. DOI:10.12688/f1000research.2-32.v2
⁴⁰Liu et al., Pediatr Int. 2013, vol. 55(2), p. 138-144.
⁴¹Waisbourd-Zinman et al., Ann Hepatol. 2017, vol. 16(3), p. 465-468.
⁴²Griffin, et al., Canadian Journal of Gastroenterology and Hepatology 2016, vol. 2016. Abstract Number: A200. Meeting Info: 2016 Canadian Digestive Diseases Week, CDDW 2016. Montreal, QC, United States. 26 Feb. 2016-29 Feb. 2016
⁴³Qiu et al., Hepatology 2017, vol. 65(5), p. 1655-1669.
⁴⁴Imagawa et al., Sci Rep. 2017, 7:41806.
⁴⁵Kang et al., J Pathol Transl Med. 2019 May 16. doi:10.4132/jptm.2019.05.03. [Epub ahead of print]
⁴⁶Takahashi et al., Eur J Gastroenterol Hepatol. 2007, vol. 19(11), p. 942-6.
⁴⁷Shimizu et al., Am J Transplant. 2011, vol. 11(2), p. 394-398.
⁴⁸Krawczyk et al., Ann Hepatol. 2012, vol. 11(5), p. 710-744.
⁴⁹Sharma et al., BMC Gastroenterol. 2018, vol. 18(1), p. 107.
⁵⁰Sattler et al., Journal of Hepatology 2017, vol. 66, No. 1, Suppl. S, pp. S177. Meeting Info.: International Liver Congress/52nd Annual Meeting of the European-Association-for-the-Study-of-the-Liver. Amsterdam, NETHERLANDS. Apr. 19-23, 2017. European Assoc Study Liver.
⁵¹Jung et al., J Pediatr Gastroenterol Nutr. 2007, vol. 44(4), p. 453-458.
⁵²Sciveres. Digestive and Liver Disease 2010, vol. 42, Supp. SUPPL. 5, pp. 5329. Abstract Number: CO18. Meeting Info: 17th National Congress SIGENP. Pescara, Italy. 7 Oct. 2010-9 Oct. 2010
⁵³Sohn et al., Pediatr Gastroenterol Hepatol Nutr. 2019, vol. 22(2), p. 201-206.
⁵⁴Ho et al., Pharmacogenet Genomics. 2010, vol. 20(1), p. 45-57.
⁵⁵Wang et al., Hepatol Res. 2018, vol. 48(7), p. 574-584.
⁵⁶Shaprio et al., J Hum Genet. 2010, vol. 55(5), p. 308-313.
⁵⁷Bounford. University of Birmingham. Dissertation Abstracts International, (2016) Vol. 75, No. 1C. Order No.: AAI10588329. ProQuest Dissertations & Theses.
⁵⁸Stolz et al., Aliment Pharmacol Ther. 2019, vol. 49(9), p. 1195-1204.
⁵⁹Jankowska et al., J Pediatr Gastroenterol Nutr. 2014, vol. 58(1), p. 92-95.
⁶⁰Kim. Journal of Pediatric Gastroenterology and Nutrition 2016, vol. 62, Supp. SUPPL. 1, pp. 620. Abstract Number: H-P-045. Meeting Info: 49th Annual Meeting of the European Society for Paediatric Gastroenterology, Hepatology and Nutrition, ESPGHAN 2016. Athens, Greece. 25 May 2016-28 May 2016.
⁶¹Pauli-Magnus et al., Hepatology 2003, vol. 38, No. 4 Suppl. 1, pp. 518A. print. Meeting Info.: 54th Annual Meeting of the American Association for the Study of Liver Diseases. Boston, MA, USA. Oct. 24-28, 2003. American Association for the Study of Liver Diseases.
⁶²Li et al., Hepatology International 2017, vol. 11, No. 1, Supp. Supplement 1, pp. S362. Abstract Number: PP0347. Meeting Info: 26th Annual Conference of the Asian Pacific Association for the Study of the Liver, APASL 2017. Shanghai, China. 15 Feb. 2017-19 Feb. 2017.
⁶³Rumbo et al., Transplantation 2018, vol. 102, No. 7, Supp. Supplement 1, pp. S848. Abstract Number: P.752. Meeting Info: 27th International Congress of The Transplantation Society, TTS 2018. Madrid, Spain. 30 Jun. 2018-5 Jul. 2018.
⁶⁴Lee et al., Pediatr Gastroenterol Hepatol Nutr. 2017, vol. 20(2), p. 114-123.
⁶⁵Sherrif et al., Liver international: official journal of the International Association for the Study of the Liver 2013, vol. 33, No. 8, pp. 1266-1270.
⁶⁶Blackmore et al., J Clin Exp Hepatol. 2013, vol. 3(2), p. 159-161.
⁶⁷Matte et al., J Pediatr Gastroenterol Nutr. 2010, vol. 51(4), p. 488-493.
⁶⁸Lin et al., Zhongguo Dang Dai Er Ke Za Zhi. 2018, vol. 20(9), p. 758-764.
⁶⁹Harmanci et al., Experimental and Clinical Transplantation 2015, vol. 13, Supp. SUPPL. 2, pp. 76. Abstract Number: P62. Meeting Info: 1st Congress of the Turkic World Transplantation Society. Astana, Kazakhstan. 20 May 2015-22 May 2015.
⁷⁰Herbst et al., Mol Cell Probes. 2015, vol. 29(5), p. 291-298.
⁷¹Moghadamrad et al., Hepatology. 2013, vol. 57(6), p. 2539-2541.
⁷²Holz et al., Zeitschrift fur Gastroenterologie 2016, vol. 54, No. 8. Abstract Number: KV275. Meeting Info: Viszeralmedizin 2016, 71. Jahrestagung der Deutschen Gesellschaft fur Gastroenterologie, Verdauungs-und Stoffwechselkrankheiten mit Sektion Endoskopie-10. Herbsttagung der Deutschen Gesellschaft fur Allgemein-und Viszeralchirurgie. Hamburg, Germany. 21 Sep. 2016-24 Sep. 2016.
⁷³Wang et al., PLoS One. 2016; vol. 11(4):e0153114.
⁷⁴Hao et al., International Journal of Clinical and Experimental Pathology 2017, vol. 10(3), p. 3480-3487.
⁷⁵Arnell et al., J Pediatr Gastroenterol Nutr. 2010, vol. 51(4), p. 494-499.
⁷⁶Sharma et al., Indian Journal of Gastroenterology 2017, vol. 36, No. 1, Supp. Supplement 1, pp. A99. Abstract Number: M-20. Meeting Info: 58th Annual Conference of the Indian Society of Gastroenterology, ISGCON 2017. Bhubaneswar, India. 14 Dec. 2017-17 Dec. 2017.
⁷⁷Beauséjour et al., Can J Gastroenterol. 2011, vol. 25(6), p. 311-314.
⁷⁸Imagawa et al., Journal of Pediatric Gastroenterology and Nutrition 2016, vol. 63, Supp. Supplement 2, pp. S51. Abstract Number: 166. Meeting Info: World Congress of Pediatric Gastroenterology, Hepatology and Nutrition 2016. Montreal, QC, Canada. 5 Oct. 2016-8 Oct. 2016.
⁷⁹Peng et al., Zhonghua er ke za zhi (Chinese journal of pediatrics) 2018, vol. 56, No. 6, pp. 440-444.
⁸⁰Tibesar et al., Case Rep Pediatr. 2014, vol. 2014: 185923.
⁸¹Ng et al., Journal of Pediatric Gastroenterology and Nutrition 2018, vol. 66, Supp. Supplement 2, pp. 860. Abstract Number: H-P-127. Meeting Info: 51st Annual Meeting European Society for Paediatric Gastroenterology, Hepatology and Nutrition, ESPGHAN 2018. Geneva, Switzerland. 9 May 2018-12 May 2018.
⁸²Wong et al., Clin Chem. 2008, vol. 54(7), p. 1141-1148.
⁸³Pauli-Magnus et al., J Hepatol. 2005, vol. 43(2), p. 342-357.
⁸⁴Jericho et al., Journal of Pediatric Gastroenterology and Nutrition. 60, vol. 3, p. 368-374.
⁸⁵Scheimann et al., Gastroenterology 2007, vol. 132, No. 4, Suppl. 2, pp. A452. Meeting Info.: Digestive Disease Week Meeting/108th Annual Meeting of the American-Gastroenterological-Association. Washington, DC, USA. May 19-24, 2007. Amer Gastroenterol Assoc; Amer Assoc Study Liver Dis; Amer Soc Gastrointestinal Endoscopy; Soc Surg Alimentary Tract.
⁸⁶Jaquotot-Haerranz et al., Rev Esp Enferm Dig. 2013, vol. 105(1), p. 52-54.
⁸⁷Khosla et al., American Journal of Gastroenterology 2015, vol. 110, No. Suppl. 1, pp. S397. Meeting Info.: 80th Annual Scientific Meeting of the American-College-of-Gastroenterology. Honolulu, HI, USA. Oct. 16-21, 2015.
⁸⁸Dröge et al., J Hepatol. 2017, vol. 67(6), p. 1253-1264.
⁸⁵Liu et al., Liver International 2010, vol. 30(6), p. 809-815.
⁹⁰Chen et al., Journal of Pediatrics 2002, vol. 140(1), p. 119-124.
⁹¹U.S. Pat. No. 9,295,677

In some embodiments, the mutation in ABCB11 is selected from A167T, G238V, V284L, E297G, R470Q, R470X, D482G, R487H, A570T, N591S, A865V, G982R, R1153C, and R1268Q.

Provided are methods of treating PFIC (e.g., PFIC-1 and PFIC-2) in a subject that includes performing an assay on a sample obtained from the subject to determine whether the subject has a mutation associated with PFIC (e.g., a ATP8B1, ABCB11, ABCB4, TJP2, NR1H4 or Myo5b mutation), and administering (e.g., specifically or selectively administering) a therapeutically effective amount of a compound of formula (I), or a pharmaceutically acceptable salt thereof, to the subject determined to have a mutation associated with PFIC. In some embodiments, the mutation is an ATP8B1 or ABCB11 mutation. For example, a mutation as provided in any one of Tables 1-4. In some embodiments, the mutation in ATP8B1 is selected from L127P, G308V, T456M, D554N, F529del, I661T, E665X, R930X, R952X, R1014X, and G1040R. In some embodiments, the mutation in ABCB11 is selected from A167T, G238V, V284L, E297G, R470Q, R470X, D482G, R487H, A570T, N591S, A865V, G982R, R1153C, and R1268Q.

Also provided are methods for treating PFIC (e.g., PFIC-1 and PFIC-2) in a subject in need thereof, the method comprising: (a) detecting a mutation associated with PFIC (e.g., a ATP8B1, ABCB11, ABCB4, TJP2, NR1H4 or Myo5b mutation) in the subject; and (b) administering to the subject a therapeutically effective amount of the formulation disclosed herein. In some embodiments, methods for treating PFIC can include administering a therapeutically effective amount of a compound of formula (I), or a pharmaceutically acceptable salt thereof, to a subject having a mutation associated with PFIC (e.g., an ATP8B1, ABCB11, ABCB4, TJP2, NR1H4 or Myo5b mutation).

In some embodiments, the mutation is an ATP8B1 or ABCB11 mutation. For example, a mutation as provided in any one of Tables 1-4. In some embodiments, the mutation in ATP8B1 is selected from L127P, G308V, T456M, D554N, F529del, I661T, E665X, R930X, R952X, R1014X, and G1040R. In some embodiments, the mutation in ABCB11 is selected from A167T, G238V, V284L, E297G, R470Q, R470X, D482G, R487H, A570T, N591S, A865V, G982R, R1153C, and R1268Q.

In some embodiments, the subject is determined to have a mutation associated with PFIC in a subject or a biopsy sample from the subject through the use of any art recognized tests, including next generation sequencing (NGS). In some embodiments, the subject is determined to have a mutation associated with PFIC using a regulatory agency-approved, e.g., FDA-approved test or assay for identifying a mutation associated with PFIC in a subject or a biopsy sample from the subject or by performing any of the non-limiting examples of assays described herein. Additional methods of diagnosing PFIC are described in Gunaydin, M. et al., Hepat Med. 2018, vol. 10, p. 95-104, incorporated by reference in its entirety herein.

In some embodiments, the treatment of PFIC (e.g., PFIC-1 or PFIC-2) decreases the level of serum bile acids in the subject. In some embodiments, the level of serum bile acids is determined by, for example, an ELISA enzymatic assay or the assays for the measurement of total bile acids as described in Danese et al., PLoS One. 2017, vol. 12(6):e0179200, which is incorporated by reference herein in its entirety. In some embodiments, the level of serum bile acids can decrease by, for example, 10% to 40%, 20% to 50%, 30% to 60%, 40% to 70%, 50% to 80%, or by more than 90% of the level of serum bile acids prior to administration of the formulation disclosed herein. In some embodiments, the treatment of PFIC includes treatment of pruritus.

In another aspect, the invention relates to a process for the preparation of the pharmaceutical formulation as disclosed herein, comprising the step of preparing a homogeneous aqueous suspension of odevixibat. In a preferred embodiment, the coating suspension is prepared by dispersing odevixibat in water by wet milling.

In a more specific embodiment, the process comprises the steps of:
a) wetting odevixibat in water using a homogenizer; and
b) dispersing the wetted odevixibat in water by wet milling, thereby obtaining a homogeneous aqueous suspension of odevixibat.

In some embodiments, odevixibat is sieved prior to the wetting of step a).

In some embodiments, the process further comprises the step of adding a film-forming polymer to the suspension. A film-forming polymer can facilitate a uniform dispersion of odevixibat in the suspension. The film-forming polymer may be added either before or after the wet milling step. In some embodiments, the wet milling is more effective in the absence of the film-forming polymer. In a preferred embodiment, therefore, the film-forming polymer is added to the homogeneous aqueous suspension of odevixibat obtained in step (b).

The homogeneity of the coating suspension may be checked either before or after addition of the film-forming polymer. When the size of the odevixibat agglomerates can be determined by optical microscopy, such as described in the experimental section, the coating suspension should not contain agglomerates larger than 200 μm. The suspension preferably does not contain agglomerates larger than 100 μm, and more preferably does not contain agglomerates larger than 50 μm. When the size of the agglomerates is determined by light scattering techniques, such as LALLS, the d₉₀ value for the particle size distribution of the coating suspension is preferably smaller than 15 μm, such as smaller than 14 μm, such as smaller than 13 μm, such as smaller than 12 μm, such as smaller than 11 μm, or such as smaller than 10 μm.

In another preferred embodiment, the coating suspension does not contain surfactants.

In another aspect, the invention relates to the formulation obtained by any of the processes disclosed herein.

### Definitions

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the term “pharmaceutically acceptable” refers to those compounds, materials, compositions and/or dosage forms that are suitable for human pharmaceutical use and that are generally safe, non-toxic and neither biologically nor otherwise undesirable.

As used herein, the term “about” refers to a value or parameter herein that includes (and describes) embodiments that are directed to that value or parameter per se. For example, description referring to “about 20” includes description of “20.” Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater.

The term “crystal modification” refers to a crystalline solid phase of an organic compound. A crystal modification can be either a solvate or an ansolvate.

The term “solvate” refers to a crystalline solid phase of an organic compound, which has solvent (i.e., solvent molecules) incorporated into its crystal structure. A “hydrate” is a solvate wherein the solvent is water.

The term “sesquihydrate” refers to a hydrate containing about 1.5 moles of water associated with the crystal per mole of organic compound (i.e., a 1.5 hydrate). As used herein, a sesquihydrate includes from about 1.2 to about 1.8, more preferably from about 1.3 to about 1.7, more preferably from about 1.4 to about 1.6 and even more preferably from about 1.45 to about 1.55 moles of water associated with each mole of odevixibat in a crystal. The amount of water calculated herein excludes water adsorbed to the surface of the crystal.

The term “mixed solvate” refers to a crystalline solid phase of an organic compound, which has two or more different solvent molecules incorporated into its crystal structure. One of the at least two solvent molecules may be water.

The term “slurry” refers to a saturated solution to which an excess of solid is added, thereby forming a mixture of solid and saturated solution.

As used herein, the term “void volumes” refers to channels, layers or other more or less isolated voids in the crystal structure.

The crystallinity of a crystalline sample of odevixibat may be measured e.g. by X-Ray Powder Diffraction (XRPD) methods or by Differential Scanning Calorimetry (DSC) methods, such as the method disclosed in the experimental section. When reference is made herein to a crystalline compound, preferably the crystallinity as measured by DSC methods is greater than about 70%, such as greater than about 80%, particularly greater than about 90%, more particularly greater than about 95%. In some embodiments, the degree of crystallinity as measured by DSC methods is greater than about 98%. In some embodiments, the degree of crystallinity as measured by DSC methods is greater than about 99%. The % crystallinity refers to the percentage by weight of the total sample mass which is crystalline.

The invention will now be described by the following examples which do not limit the invention in any respect. All cited documents and references mentioned herein are incorporated by reference in their entireties.

### Abbreviations

DMF dimethylformamide
DMSO dimethyl sulfoxide
EtOH ethanol
MeOH methanol
RH relative humidity
2-PrOH 2-propanol

### Experimental Methods

X-Ray Powder Diffraction (XRPD) Analysis Analyses were performed at 22° C. on a PANalytical X'Pert Pro diffractometer equipped with a Cu long fine focus X-ray tube and a PIXcel detector. Automatic divergence and anti-scatter slits were used together with 0.02 rad Soller slits and a Ni-filter. Dry samples were smeared onto cut Silicon Zero Background Holders (ZBH) and analysed between 2-40° in 2-theta with an analysis time of 17 minutes. All slurry samples were dripped on tempered porous Alumina filter substrates and analysed twice as they dried, first with a one minute 16-second scan (2-30° in 2-theta) and then a 7-minute scan (2-30° in 2-theta). A final 17-minute scan was performed when the sample had dried for several hours.

The samples were spun during analysis in order to increase the randomness of the samples. The following experimental settings were used:
Tube tension and current: 40 kV, 50 mA
Wavelength alpha1 (CuKα1): 1.5406 Å
Wavelength alpha2 (CuKα2): 1.5444 Å
Wavelength alpha1 and alpha2 mean (CuKα): 1.5418 Å

It is known in the art that an X-ray powder diffraction pattern may be obtained having one or more measurement errors depending on measurement conditions (such as equipment, sample preparation or machine used). In particular, it is generally known that intensities in an XRPD pattern may fluctuate depending on measurement conditions and sample preparation. For example, persons skilled in the art of XRPD will realise that the relative intensities of peaks may vary according to the orientation of the sample under the test and on the type and setting of the instrument used. The skilled person will also realise that the position of reflections can be affected by the precise height at which the sample sits in the diffractometer and the zero calibration of the diffractometer. The surface planarity of the sample may also have a small effect. Hence a person skilled in the art will appreciate that the diffraction pattern presented herein is not to be construed as absolute and any crystalline form that provides a powder diffraction pattern substantially identical to those disclosed herein fall within the scope of the present disclosure (for further information, see R. Jenkins and R. L. Snyder, “Introduction to X-ray powder diffractometry”, John Wiley & Sons, 1996).

### Differential Scanning Calorimetry (DSC)

Experiments were performed using a TA Instruments Q2000 Differential Scanning Calorimeter. The DCS crucible used was a TZero aluminum pan with pinhole (diameter≥0.2 mm) in the lid. A dry nitrogen purge at a constant flow rate of 50 mL/min was maintained in the DSC cell throughout the measurement.

### EXAMPLES

### Example 1

### Preparation of the Formulation (Small Scale)

Microcrystalline cellulose spheres were coated with one of two different coating suspensions of odevixibat, as indicated in Table 5 below, to obtain particles containing either 0.5% w/w or 1.5% w/w odevixibat.

TABLE 5

| | Amount | Amount |
|---|---|---|
| Ingredient | (g/batch) | (g/batch) |
| Core: | | |
| Microcrystalline cellulose spheres 700 | 1500 | 1500 |
| (Vivapur ® MCC sphere 700) | | |
| Coating: | | |
| Odevixibat | 7.5 | 22.5 |
| Hypromellose 3 mPa · s (Methocel ® E3 | 30.0 | 90.0 |
| premium) | | |
| Purified water^{a} | 337.5 | 1012.5 |
| Total (coated particles) | 1537.5 | 1612.5 |

^{a}Purified water is removed during the coating and drying process.

Crystalline odevixibat was used. Typical values for the particle size distribution of the crystalline material were d₁₀=0.9 μm, d₅₀=4 μm and d₉₀=20 μm, wherein d₁₀, d₅₀ and d₉₀ are defined as the diameters where 10%, 50% and 90%, respectively, of the particle population lies below these values.

### Coating Suspension

The coating suspension containing odevixibat drug substance was prepared in three steps:
a) Odevixibat suspension: odevixibat drug substance was sieved through a 0.5 mm sieve, followed by wetting in a small amount of the water using a homogenizer (Ultra Turrax T25; 15 minutes at approximately 6600-7000 rpm). The resulting wetted odevixibat drug substance was then dispersed in water by means of a colloid mill (IKA Magic Lab M KO or MK modules, 14600 rpm for 20 minutes, gap size 1.5 rotation) until the level of agglomerates met the in-process control acceptance limits.
b) Hypromellose dispersion: Hypromellose (3 mPa·s) was dispersed in hot water with mixing, and the resultant dispersion was cooled to room temperature.
c) Odevixibat coating suspension: The hypromellose dispersion was added to the odevixibat suspension in the colloid mill and the suspension was mixed for 4 minutes at 10000 rpm. Final mixing was continued at low speed using a magnetic stirrer. The odevixibat coating suspension was filtered through a 0.5 mm sieve before use in the coating process.

The dispersion of odevixibat in the coating suspension was monitored by optical microscopy, using a method based on European Pharmacopoeia 9.0, monograph 2.9.37, which was adjusted to be applicable for the odevixibat coating suspension. A Leica DMLB microscope equipped with a Leica DMC 2900 digital camera was used, and an objective with 10×magnification.

Samples were prepared by placing a small droplet of the coating suspension (using a Pasteur pipette) on a blank objective glass on top of a grid counting chamber of 4×4 test fields. A cover glass (about 18×18 mm, the same size as the grid) was placed on the droplet and slightly pressed on the centre to get a thin, even sample. The diameter of the sample was comparable with the size of the cover glass.

The objective was set with magnification×10 and the scale bar was adjusted to 100 μm. Five replicates were scanned. The size of any agglomerates was checked by comparing them against the scale bar in four predetermined test fields for each replicate. The total number of agglomerates was calculated from 5 replicates×4 test fields, i.e. in total 20 test fields. The coating suspension was accepted if the 20 test fields did not contain more than 5 agglomerates μm, and no agglomerates≥200 μm.

### Coating Process

Microcrystalline cellulose (MCC) spheres were coated using the odevixibat coating suspension in a fluid bed coater with Wurster insert. The amount of coating suspension on the MCC spheres is determined by weighing. The coated particles were sieved through a 0.5 mm and 1.25 mm sieve, respectively, in order to remove fine particles as well as twins. The particles were then transferred to bulk containers and handled as a drug product intermediate.

### Capsule Filling

The calculated amount of particles required for each unit dose were filled into hard hydroxypropyl methylcellulose (HPMC) capsules (Size 0 or Size 3) using an automatic capsule filler, to provide four different strengths: 200, 400, 600 and 1200 μg.

The 200 and 600 μg strengths are Size 0 white capsules containing 40 mg of particles having an odevixibat concentration of 0.5% w/w and 1.5% w/w, respectively. These strengths will be used for patients with a weight range of 5.0 kg to<19.5 kg in the low—(40 μg/kg) and high—(120 μg/kg) dose groups of the Phase 3 clinical studies. The Size 0 capsules are designed to be opened so that the contents can be sprinkled onto a food vehicle for administration. They are not intended to be swallowed intact.

The 400 μg and 1200 μg strengths are Size 3 white capsules containing 80 mg of particles having an odevixibat concentration of 0.5% w/w and 1.5% w/w, respectively. These strengths will be used for patients with a weight range of 19.5 kg to>55.5 kg in the low—(40 μg/kg) and high—(120 μg/kg) dose groups of the Phase 3 clinical studies. The Size 3 capsules are intended to be swallowed intact.

The fill weight, the amounts of odevixibat and other ingredients and the capsule size for the different capsule strengths are shown in Table 6 below.

TABLE 6

| | Strength | | | |
|---|---|---|---|---|
| | 200 | 400 | 600 | 1200 |
| COMPONENT | μg | μg | μg | μg |
| odevixibat concentration of | 0.5% w/w | | 1.5% w/w | |
| particles | | | | |
| Fill weight (mg) (theoretical) | 40 | 80 | 40 | 80 |
| Particles | | | | |
| Microcrystalline cellulose spheres | 39 | 78 | 37 | 74 |
| 700 (Vivapur ® MCC sphere 700) | | | | |
| Odevixibat | 0.200 | 0.400 | 0.600 | 1.200 |
| Hypromellose 3 mPa · s | 0.8 | 1.6 | 2.4 | 4.8 |
| (Methocel ® E3 Premium) | | | | |
| Capsule | | | | |
| Hypromellose capsule, white | Size 0 | Size 3 | Size 0 | Size 3 |
| (Vcaps ® Plus) | | | | |

### Example 2

### Preparation of the Formulation (Larger Scale)

Microcrystalline cellulose spheres were coated with one of two different coating suspensions of odevixibat, as indicated in Table 7 below, to obtain particles containing either 0.5% w/w or 1.5% w/w odevixibat.

TABLE 7

| | Amount | Amount |
|---|---|---|
| Ingredient | (kg/batch) | (kg/batch) |
| Core: | | |
| Microcrystalline cellulose spheres 700 | 14.625 | 13.875 |
| (Vivapur ® MCC sphere 700) | | |
| Coating: | | |
| Odevixibat | 0.075 | 0.225 |
| Hypromellose 3 mPa · s (Methocel ® E3 | 0.300 | 0.900 |
| premium) | | |
| Purified water^{a} | 3.375 | 10.125 |
| Total (coated particles) | 15.000 | 15.000 |

^{a}Purified water is removed during the coating and drying process.

Crystalline odevixibat was used. Typical values for the particle size distribution of the crystalline material were d₁₀=0.9 μm, d₅₀=4 μm and d₉₀=20 μm, wherein d₁₀-, d₅₀ and d₉₀ are defined as the diameters where 10%, 50% and 90%, respectively, of the particle population lies below these values.

### Coating Suspension

The coating suspension containing odevixibat drug substance was prepared in three steps:
a) odevixibat suspension: odevixibat drug substance was wetted in a small amount of the water using a homogenizer (Ultra Turrax T25; 15 minutes at approximately 6600-7000 rpm). The resulting wetted odevixibat drug substance was then dispersed in water by means of a colloid mill (IKA Magic Lab MKO or MK modules, 14600 rpm for 20 minutes, gap size 1.5 rotation) until the level of agglomerates met the in-process control acceptance limits, i.e. d₉₀<12 μm (as determined by low-angle laser light scattering (LALLS)).
b) hypromellose dispersion: Hypromellose (3 mPa·s) was dispersed in hot water with mixing, and the resultant dispersion was cooled to room temperature.
c) odevixibat coating suspension: The hypromellose dispersion was added to the odevixibat suspension and the suspension was mixed. Final mixing was continued at low speed using a stirrer. The odevixibat coating suspension was filtered through a 0.5 mm sieve before use in the coating process.

### Coating Process

The obtained odevixibat coating suspension was used for coating microcrystalline cellulose (MCC) spheres in accordance with the coating process described in Example 1.

### Capsule Filling

Capsules were prepared in accordance with Example 1. The fill weight, the amounts of odevixibat and other ingredients and the capsule size for the different capsule strengths were as presented in Table 5 above.

### Example 3

### Preparation of Crystal Modification 1

Absolute alcohol (100.42 kg) and crude odevixibat (18.16 kg) were charged to a 250-L GLR with stirring under nitrogen atmosphere. Purified water (12.71 kg) was added and the reaction mass was stirred under nitrogen atmosphere at 25±5° C. for 15 minutes. Stirring was continued at 25±5° C. for 3 to 60 minutes, until a clear solution had formed. The solution was filtered through a 5.0 μSS cartridge filter, followed by a 0.2 μPP cartridge filter and then transferred to a clean reactor. Purified water (63.56 kg) was added slowly over a period of 2 to 3 hours at 25±5° C., and the solution was seeded with crystal modification 1 of odevixibat. The solution was stirred at 25±5° C. for 12 hours. During this time, the solution turned turbid. The precipitated solids were filtered through centrifuge and the material was spin dried for 30 minutes. The material was thereafter vacuum dried in a Nutsche filter for 12 hours. The material was then dried in a vacuum tray drier at 25±5° C. under vacuum (550 mm Hg) for 10 hours and then at 30±5° C. under vacuum (550 mm Hg) for 16 hours. The material was isolated as an off-white crystalline solid. The isolated crystalline material was milled and stored in LDPE bags.

An overhydrated sample was analyzed with XRPD and the diffractogram is shown in FIG. 2. Another sample was dried at 50° C. in vacuum and thereafter analysed with XRPD. The diffractogram of the dried sample is shown in FIG. 1.

The diffractograms for the drying of the sample are shown in FIGS. 3 and 4 for 2θ ranges 5-13° and 18-25°, respectively (overhydrated sample at the bottom and dry sample at the top).

### Example 4

### Preparation of Crystal Modification 2 from Ethanol and Water

105.9 mg of odevixibat were weighed into a 1 mL Chromacol vessel. A magnetic stir bar and 1.0 mL of an ethanol:water 70:30% v/v mixture were added and the vessel was closed with a crimped cap. The resulting slurry was then left stirred at 25° C. for 1 week.

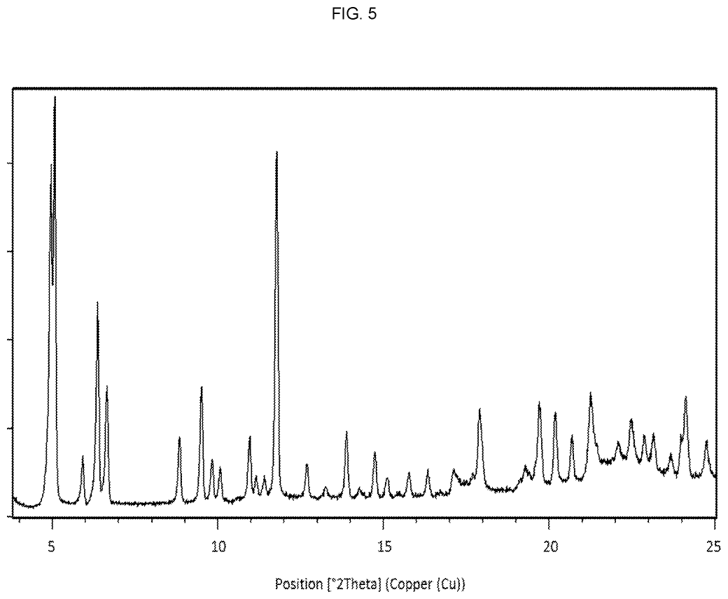

The wet sample was analyzed with XRPD and the diffractogram is shown in FIG. 5. Upon drying of the sample, it transformed into crystal modification 1.

### Example 5

### Determination of Crystalline Fraction by Differential Scanning Calorimetry

This method quantifies the crystalline fraction of odevixibat drug substance in partially crystalline samples. The quantification is based on the assumption that partially crystalline samples are binary mixtures of the crystalline hydrate and the amorphous phase of odevixibat. The crystalline fraction is quantified based on the melting enthalpy of an anhydrous form. This anhydrous form is the dehydrated hydrate which spontaneously and reproducibly forms by drying the hydrate at elevated temperature.

5-6 mg of a sample of a crystalline or partially crystalline sample of odevixibat was accurately weighed into a DSC crucible which was then closed with a perforated lid using a suitable press. The total weight of the DSC crucible (pan+lid+sample) was noted and the total weight of the crucible was again determined after the DSC test. The weight loss during the DSC test must not be more than 5%.

The DSC test consists of three cycles:
Cycle 1: an increase in temperature from 20° C. to 120° C. at a scanning rate of 5° C./min;
Cycle 2: a decrease in temperature from 120° C. to 80° C. at a scanning rate of 10° C./min; and
Cycle 3: an increase in temperature from 80° C. to 200° C. at a scanning rate of 10° C./min.

The first scan cycle dries the sample and thereby converts the hydrate form into a dehydrated hydrate (an anhydrous form). In the second scan cycle, the sample is cooled down to obtain a stable baseline in the subsequent heat-up for signal integration. The melting enthalpy is determined in the third scan cycle, where the sample is heated through the melting of the anhydrous form.

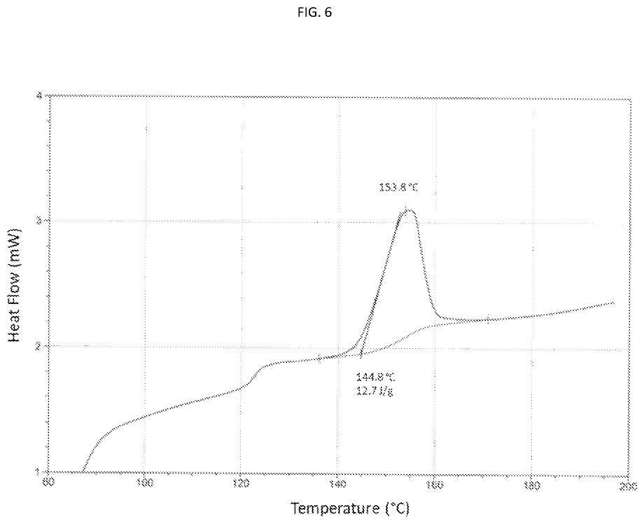

The endothermic event due to melting appears in the temperature range of 140-165° C. The peak must be integrated over a sigmoidal tangent baseline using the Sig Tangent integration function of the TA Universal Analysis software. The integration should start at a temperature between 130° C. and 140° C., and end at a temperature between 165° C. and 175° C., depending on the actual baseline. The glass transition of the amorphous part may appear in the temperature range of 120-130° C., depending on the actual amorphous fraction (see FIG. 6 for an example). If an irregular baseline does not allow the integration, it should be assessed whether the drying of the sample was incomplete.

The evaluation of the melting enthalpy is done by using the dry weight of the sample, which is obtained by subtracting the total weight of the DSC crucible (pan+lid+sample) after the DSC test from the total weight of the crucible before the test. The percent weight loss during the DSC scan, which is the difference between the initial weight and the dry weight divided by the initial weight, must not be more than 5%; otherwise the crystalline content of the sample cannot be calculated. The crystalline fraction expressed in weight percent is to be calculated from the melting enthalpy (ΔHₛₐₘₚₗₑ) based on the following formula. The value shall be given on an integer number.

$$\% crystalline content = \frac{Δ {H}_{sample} + 1.1626}{0.2815}$$

### Example 6

### Homogeneity Monitored by LALLS

The homogeneity of odevixibat coating suspensions was studied using low-angle laser light scattering (LALLS). Three odevixibat coating suspensions intended for particles containing 0.5% w/w odevixibat were produced by dispersing odevixibat drug substance (16 g) in water (200 g) with an Ultra Turrax homogenizer for 7 minutes. When the drug substance was dispersed, the homogenizer was run for an additional 8 minutes. The homogenizer was then rinsed with water (216 g), which was added to the suspension.

The odevixibat suspensions were then dispersed using a wet mill (IKA Magic Lab and MK module), using the settings presented in Table 8. Methocel E3 was dispersed in hot water (85-90° C.) while mixing with an overhead stirrer and then cooled to room temperature. The concentration was adjusted to 17.4% w/w by the addition of water.

368 g of the Methocel gel was charged to the odevixibat suspension and mixed using the wet mill for another 4 minutes at 10000 rpm. The temperature of the suspension was checked during the process. The homogeneity of the odevixibat suspension was monitored with LALLS after 0, 5, 10, 15 and 20 minutes recirculation time. The data are presented in Table 9.

TABLE 8

| Suspension No. | 1 | 2 | 3 |
|---|---|---|---|
| Dispersion (Ultra Turrax) | | | |
| Time (min) | 15 | 15 | 15 |
| Speed (rpm) | 6800-8000 | 7600-8000 | 6600-8000 |
| Dispersion (Wet mill) | | | |
| Gap (rotations) | 1.5 | 1.5 | 1.0 |
| Time (min) | 20 | 20 | 20 |
| Speed (rpm) | 14600 | 14600 | 14600 |
| Cooling (MK module) | | | |
| Set point (° C.) | N/A | 25 | 10 |
| Temperature (° C.) of coating | 72 | 50 | 55 |
| suspension after 20 min wet milling | | | |

TABLE 9

| Suspension | Recirculation | LALLS (μm) | | | |
|---|---|---|---|---|---|
| No. | time (min) | d₁₀ | d₅₀ | d₉₀ | Comments |
| 1 | 0 | 1.5 | 5.5 | 17.9 | |
| | 5 | 1.1 | 3.9 | 10.5 | |
| | 10 | 1.2 | 3.9 | 9.3 | |
| | 15 | 2.4 | 5.2 | 11.3 | |
| | 20 | 1.8 | 4.1 | 8.8 | Sample taken after |
| | | | | | addition of Methocel |
| | 20 | 1.6 | 3.9 | 8.7 | Sample taken after 5 |
| | | | | | days storage at room |
| | | | | | temperature with |
| | | | | | magnetic stirring |
| 2 | 0 | 1.5 | 6.5 | 29.1 | |
| | 5 | 1.2 | 4.3 | 11.4 | |
| | 10 | 1.1 | 3.8 | 9.3 | |
| | 15 | 1.1 | 3.6 | 8.5 | |
| | 20 | 1.0 | 3.5 | 8.2 | Sample taken when |
| | | | | | wet milling was |
| | | | | | finished |
| | 20 | 1.1 | 3.7 | 8.6 | Sample taken after |
| | | | | | addition of Methocel |
| 3 | 0 | n.d. | n.d. | n.d. | |
| | 5 | 0.9 | 3.4 | 8.8 | |
| | 10 | 0.9 | 3.2 | 7.7 | |
| | 15 | 1.1 | 3.7 | 8.5 | |
| | 20 | 1.1 | 3.8 | 8.9 | Sample taken when |
| | | | | | wet milling was |
| | | | | | finished |
| | 20 | 1.0 | 3.4 | 8.0 | Sample taken after |
| | | | | | addition of Methocel |

### Example 7

### Content Uniformity

Pellets of two different strengths, 0.5% w/w and 1.5% w/w, were prepared as described in Example 1. The amount of odevixibat in a capsule was determined for 30 capsules, using reversed-phase high-performance liquid chromatography (RP-HPLC). Mobile phase: 40:60 acetonitrile:acetate buffer pH 5.5; flow rate 1.5 mL/min; column: Zorbax SB-CN (50×4.6 mm, 3.5 μm). The assayed amount of odevixibat and the content uniformity are presented in Table 10.

TABLE 10

| | 0.5% | 1.5% | 0.5% |
|---|---|---|---|
| Odevixibat concentration in particles | w/w | w/w | w/w |
| Assay (mg/g) | 4.98 | 14.2 | 4.88 |
| Content Uniformity: RSD % (n = 30) | 1.6 | 0.8 | 1.4 |

### Example 8

### Stability Testing at Low pH

The compatibility between particles coated with odevixibat and yoghurt, apple sauce, or fruit purée was evaluated by sprinkling about 40 mg of coated particles containing 0.5% w/w odevixibat (corresponding to the contents one 200 μg capsule) onto 1 tablespoon of the food and determining recovery over a period of 120 minutes. The recovery of odevixibat was determined using a reversed-phase high-performance liquid chromatography (RP-HPLC) method. Mobile phase: 40:60 acetonitrile:acetate buffer pH 5.5; flow rate 1.5 mL/min; column: Zorbax SB-CN (50×4.6 mm, 3.5 μm).

The recovery for all food samples ranged between 95% to 101% with no change over time. Visual inspection concluded that the particles were intact for up to 6 hours for all samples; no colour differences and no dissolved particles were observed. The results verify that patients who sprinkle the particles onto food will receive the intended dose. A summary of the foods tested is presented in Tablell, and the results of the recovery testing are presented in Table 12 (apple sauce), Table 13 (yoghurt smoothie), and Table 14 (fruit purée).

TABLE 11

| Food | Brand | pH | Ingredients |
|---|---|---|---|
| Apple sauce | Eldorado | 3.7 | Apple 92%, sugar 7%, |
| | | | antioxidant (ascorbic acid), |
| | | | preservatives (E202) |
| Yoghurt | Semper | 3.9 | Banana 42%, strawberry 30%, |
| smoothie | | | yoghurt (heat treated) 20%, |
| | | | water, corn starch, chokeberry |
| | | | juice concentrate, lemon juice |
| | | | concentrate, antioxidant |
| | | | (ascorbic acid) |
| Fruit purée | Semper | 3.9 | Water, orange juice (from |
| | | | concentrate) 23%, apple 20%, |
| | | | banana 15%, rice starch, |
| | | | vitamin C, iron |

TABLE 12

| | Added | Nominal | | | |
|---|---|---|---|---|---|
| | particle | amount of | Assay for | | |
| Time | weight | odevixibat | odevixibat | Recovery | Mean |
| (min) | (mg) | (μg) | (μg) | (%) | (%) |
| 0 | 39.58 | 201.07 | 199.35 | 99.1 | 99 |
| | 42.35 | 215.14 | 212.28 | 98.7 | |
| 15 | 40.37 | 205.08 | 187.29 | 91.3 | 96 |
| | 40.37 | 205.08 | 205.37 | 100.1 | |
| 30 | 41.21 | 209.35 | 206.45 | 98.6 | 98 |
| | 39.97 | 203.05 | 197.70 | 97.4 | |
| 60 | 40.50 | 205.74 | 198.14 | 96.3 | 97 |
| | 40.20 | 204.22 | 199.99 | 97.9 | |
| 120 | 39.61 | 201.22 | 199.22 | 99.0 | 98 |
| | 40.12 | 203.81 | 199.65 | 98.0 | |

TABLE 13

| | Added | Nominal | | | |
|---|---|---|---|---|---|
| | particle | amount of | Assay for | | |
| Time | weight | odevixibat | odevixibat | Recovery | Mean |
| (min) | (mg) | (μg) | (μg) | (%) | (%) |
| 0 | 42.07 | 213.72 | 213.60 | 99.9 | 99 |
| | 42.38 | 215.29 | 209.17 | 97.2 | |
| 15 | 41.24 | 209.50 | 206.01 | 98.3 | 97 |
| | 40.42 | 205.33 | 195.58 | 95.2 | |
| 30 | 40.70 | 206.76 | 199.48 | 96.5 | 96 |
| | 41.96 | 213.16 | 203.30 | 95.4 | |
| 60 | 40.39 | 205.18 | 197.33 | 96.2 | 96 |
| | 39.38 | 200.05 | 192.06 | 96.0 | |
| 120 | 40.99 | 208.23 | 199.05 | 95.6 | 96 |
| | 39.42 | 200.25 | 192.38 | 96.1 | |

TABLE 14

| | Added | Nominal | | | |
|---|---|---|---|---|---|
| | particle | amount of | Assay for | | |
| Time | weight | odevixibat | odevixibat | Recovery | Mean |
| (min) | (mg) | (μg) | (μg) | (%) | (%) |
| 0 | 43.57 | 221.34 | 188.05 | 85.0 | 95 |
| | 37.79 | 191.97 | 200.25 | 104.3 | |
| 15 | 39.74 | 201.88 | 194.86 | 96.5 | 101 |
| | 39.34 | 199.85 | 209.63 | 104.9 | |
| 30 | 41.72 | 211.94 | 203.61 | 96.1 | 101 |
| | 41.77 | 212.19 | 226.27 | 106.6 | |
| 60 | 42.25 | 214.63 | 208.02 | 96.9 | 96 |
| | 43.39 | 220.42 | 210.74 | 95.6 | |
| 120 | 39.05 | 198.37 | 196.18 | 98.9 | 97 |
| | 40.33 | 204.88 | 196.19 | 95.8 | |

The primary degradation pathway expected for odevixibat particles, when mixed with the specified food vehicles for administration, is acidic hydrolysis of the dipeptide moiety. To evaluate the chemical stability of the odevixibat particles, 1 mL of phosphate buffer pH 3.0 was added to about 200 mg of particles containing 0.5% w/w odevixibat (i.e., the content of five 200 μg capsules) and left at room temperature for 2 hours. No degradation was observed.

### Example 9

### Long-Term Stability Testing

Odevixibat capsules of size 0 and size 3, prepared in accordance with Example 1, were stored in a HDPE bottle with an HDPE cap and kept at 25° C. and 60% relative humidity as the long-term storage condition, and at 40° C. and 75% relative humidity as the accelerated condition. The amounts of odevixibat, related substances and water were determined after 1, 3, 6, 9 and 12 months for samples stored at 25° C./60% RH, and after 1, 3, and 6 months for samples stored at 40° C./75% RH. The results for capsules of strength 200 μg (Size 0) are presented in Table 15; for capsules of strength 600 μg (Size 0) in Table 16; for capsules of strength 400 μg (Size 3) in Table 17; and for capsules of strength 1200 μg (Size 3) in Table 18.

TABLE 15
Stability data for capsules of strength 200 μg.

| | Acceptance | Storage | Storage period (months) | | | | | |
|---|---|---|---|---|---|---|---|---|
| Test | Criteria | Conditions | 0 | 1 | 3 | 6 | 9 | 12 |
| Description | White oblong | 25° C./60% RH | Conforms | Conforms | Conforms | Conforms | Conforms | Conforms |
| | hard capsule | 40° C./75% RH | | Conforms | Conforms | Conforms | | |
| | containing | | | | | | | |
| | white to off- | | | | | | | |
| | white pellets | | | | | | | |
| Assay | 180-220 | 25° C./60% RH | 207 | 205 | 206 | 204 | 209 | 207 |
| | μg/capsule | 40° C./75% RH | | 206 | 206 | 205 | | |
| Related | ≤4.0% | 25° C./60% RH | 0.53 | 0.51 | 0.48 | 0.47 | 0.55 | 0.57 |
| Substances, | | 40° C./75% RH | | 0.44 | 0.63 | 0.90 | | |
| Total | | | | | | | | |
| Dissolution | Q = 75% at | 25° C./60% RH | 104 | 101 | 102 | 102 | 106 | 106 |
| | 45 mins | 40° C./75% RH | | 99 | 102 | 101 | | |
| Water | Report (%) | 25° C./60% RH | 3.3 | NT | NT | 3.5 | NT | 4.3 |
| Content | | 40° C./75% RH | | NT | NT | 4.5 | | |

TABLE 16
Stability data for capsules of strength 600 μg.

| | Acceptance | Storage | Storage period (months) | | | | | |
|---|---|---|---|---|---|---|---|---|
| Test | Criteria | Conditions | 0 | 1 | 3 | 6 | 9 | 12 |
| Description | White oblong | 25° C./60% RH | Conforms | Conforms | Conforms | Conforms | Conforms | Conforms |
| | hard capsule | 40° C./75% RH | | Conforms | Conforms | Conforms | | |
| | containing | | | | | | | |
| | white to off- | | | | | | | |
| | white pellets | | | | | | | |
| Assay | 540-660 | 25° C./60% RH | 576 | 600 | 596 | 597 | 603 | 599 |
| | μg/capsule | 40° C./75% RH | | 599 | 612 | 601 | | |
| Related | ≤4.0% | 25° C./60% RH | 0.53 | 0.52 | 0.47 | 0.44 | 0.43 | 0.41 |
| Substances, | | 40° C./75% RH | | 0.45 | 0.48 | 0.73 | | |
| Total | | | | | | | | |
| Dissolution | Q = 75% at | 25° C./60% RH | 99 | 99 | 100 | 97 | 102 | 106 |
| | 45 mins | 40° C./75% RH | | 100 | 99 | 96 | | |
| Water | Report (%) | 25° C./60% RH | 3.4 | NT | NT | 3.3 | NT | 4.4 |
| Content | | 40° C./75% RH | | NT | NT | 4.4 | | |

TABLE 17
Stability data for capsules of strength 400 μg.

| | Acceptance | Storage | Storage period (months) | | | | | |
|---|---|---|---|---|---|---|---|---|
| Test | Criteria | Conditions | 0 | 1 | 3 | 6 | 9 | 12 |
| Description | White oblong | 25° C./60% RH | Conforms | Conforms | Conforms | Conforms | Conforms | Conforms |
| | hard capsule | 40° C./75% RH | | Conforms | Conforms | Conforms | | |
| | containing | | | | | | | |
| | white to off- | | | | | | | |
| | white pellets | | | | | | | |
| Assay | 360-440 | 25° C./60% RH | 397 | 395 | 397 | 400 | 396 | 399 |
| | μg/capsule | 40° C./75% RH | | 394 | 392 | 398 | | |
| Related | ≤4.0% | 25° C./60% RH | 0.63 | 0.51 | 0.47 | 0.57 | 0.57 | 0.59 |
| Substances, | | 40° C./75% RH | | 0.42 | 0.55 | 0.83 | | |
| Total | | | | | | | | |
| Dissolution | Q = 75% at | 25° C./60% RH | 100 | 99 | 99 | 99 | 102 | 99 |
| | 45 mins | 40° C./75% RH | | 100 | 101 | 99 | | |
| Water | Report (%) | 25° C./60% RH | 3.7 | NT | NT | 3.1 | NT | 4.2 |
| Content | | 40° C./75% RH | | NT | NT | 4.3 | | |

TABLE 18
Stability data for capsules of strength 1200 μg.

| | Acceptance | Storage | Storage period (months) | | | | | |
|---|---|---|---|---|---|---|---|---|
| Test | Criteria | Conditions | 0 | 1 | 3 | 6 | 9 | 12 |
| Description | White oblong | 25° C./60% RH | Conforms | Conforms | Conforms | Conforms | Conforms | Conforms |
| | hard capsule | 40° C./75% RH | | Conforms | Conforms | Conforms | | |
| | containing | | | | | | | |
| | white to off- | | | | | | | |
| | white pellets | | | | | | | |
| Assay | 1080-1320 | 25° C./60% RH | 1191 | 1194 | 1174 | 1169 | 1196 | 1175 |
| | μg/capsule | 40° C./75% RH | | 1200 | 1192 | 1191 | | |
| Related | ≤4.0% | 25° C./60% RH | 0.53 | 0.51 | 0.48 | 0.46 | 0.44 | 0.51 |
| Substances, | | 40° C./75% RH | | 0.55 | 0.51 | 0.63 | | |
| Total | | | | | | | | |
| Dissolution | Q = 75% at | 25° C./60% RH | 99 | 97 | 99 | 99 | 98 | 98 |
| | 45 mins | 40° C./75% RH | | 101 | 100 | 98 | | |
| Water | Report (%) | 25° C./60% RH | 3.5 | NT | NT | 3.0 | NT | 4.1 |
| Content | | 40° C./75% RH | | NT | NT | 4.3 | | |

### Example 10

### Blend Uniformity of Pellets

Particles of two different strengths, 0.5% w/w and 1.5% w/w, were prepared as described in Example 2. The sieved pellets were collected in a 55L drum, lined with a PE bag. The pellets were sampled from 10 different locations of the drum with a sampling thief tip of 0.25 mL. The average sample from each location was 80 mg, corresponding to the content of two Size 0 capsules of 200 μg or 600 μg, or one Size 3 capsule of 400 μg or 1200 μg. The content of odevixibat was determined by RP-UPLC: Mobile phase A: 80:20 ammonium acetate buffer pH 5.7/acetonitrile; Mobile phase B: 20:80 ammonium acetate buffer pH 5.7/acetonitrile; flow rate 0.40 mL/min; column: Waters Acquity BEH C8 100×2.1 mm, 1.7 mm; Gradient: 0 min: 60% A: 40% B, 12 min: 20% A: 80% B, 13.5 min: 20% A: 80% B, 13.6 min: 60% A: 40% B, 15 min: 60% A: 40% B. The assayed amount of odevixibat and the content uniformity are presented in Table 19.

TABLE 19

| | Assay for odevixibat | |
|---|---|---|
| | (% of Label Claim) | |
| Sample | 0.5% w/w | 1.5% w/w |
| 1 | 101.7 | 99.5 |
| 2 | 97.6 | 101.7 |
| 3 | 98.8 | 101.1 |
| 4 | 100.8 | 101.5 |
| 5 | 100.4 | 97.7 |
| 6 | 99.7 | 98.5 |
| 7 | 100.4 | 102.7 |
| 8 | 99.5 | 103.5 |
| 9 | 98.4 | 102.5 |
| 10 | 99.5 | 100.6 |
| Mean | 99.7 | 100.9 |
| Min | 97.6 | 97.7 |
| Max | 101.7 | 103.5 |
| RSD (%) | 1.2 | 1.9 |

1.-33. (canceled)

34. A pharmaceutical formulation of odevixibat comprising a plurality of particles, wherein each particle comprises:
a) a core, and
b) a coating layer surrounding the core, wherein the coating layer comprises odevixibat, or a pharmaceutically acceptable salt thereof, and a film-forming polymer; and wherein the coating layer comprises odevixibat agglomerates having a d₉₀ particle size distribution of less than 15 μm.

35. The formulation according to claim 34, wherein the plurality of particles is contained within a capsule.

36. The formulation according to claim 35, wherein each particle comprises odevixibat, or a pharmaceutically acceptable salt thereof, in an amount of from about 0.1% w/w to about 5.0% w/w based on the total weight of the particle.

37. The formulation according to claim 36, wherein the capsule contains from about 20 to about 100 mg of the particles.

38. The formulation according to claim 35, wherein each particle comprises odevixibat, or a pharmaceutically acceptable salt thereof, in an amount of about 0.5% w/w based on the total weight of the particle.

39. The formulation according to claim 38, wherein the capsule contains about 80 mg of the particles.

40. The formulation according to claim 35, wherein each particle comprises odevixibat, or a pharmaceutically acceptable salt thereof, in an amount of about 1.5% w/w based on the total weight of the particle.

41. The formulation according to claim 40, wherein the capsule contains about 80 mg of the particles.

42. The formulation according to claim 35, wherein the capsule contains about 200 μg, about 400 μg, about 600 μg, or about 1200 μg of odevixibat, or a pharmaceutically acceptable salt thereof.

43. The formulation according to claim 34, wherein each particle comprises odevixibat, or a pharmaceutically acceptable salt thereof, in an amount of about 0.5% w/w based on the total weight of the particle.

44. The formulation according to claim 43, wherein the plurality of particles is contained within a capsule designed to be opened.

45. The formulation according to claim 44, wherein the capsule contains about 40 mg of the particles.

46. The formulation according to claim 34, wherein each particle comprises odevixibat, or a pharmaceutically acceptable salt thereof, in an amount of about 1.5% w/w based on the total weight of the particle.

47. The formulation according to claim 46, wherein the plurality of particles is contained within a capsule designed to be opened.

48. The formulation according to claim 47, wherein the capsule contains about 40 mg of the particles.

49. The formulation according to claim 34, wherein the core comprises microcrystalline cellulose.

50. The formulation according to claim 34, wherein the core does not contain odevixibat, or a pharmaceutically acceptable salt thereof.

51. The formulation according to claim 34, wherein odevixibat is present as a crystalline hydrate of odevixibat.

52. The formulation according to claim 34, wherein odevixibat is present as crystal modification 1 of odevixibat.

53. The formulation according to claim 52, wherein crystal modification 1 of odevixibat has an X-ray powder diffraction (XRPD) pattern, obtained with CuKα1-radiation, with at least specific peaks at °2θ positions 5.6±0.2, 6.7±0.2 and/or 12.1±0.2.

54. A method for treating a cholestatic liver disease comprising administering to a subject in need of such treatment the pharmaceutical formulation of claim 1.

55. The method of claim 54, wherein the subject is administered from about 40 μg/kg to about 120 μg/kg of odevixibat, or a pharmaceutically acceptable salt thereof, per dose of the pharmaceutical formulation.

56. The method of claim 54, wherein the subject is administered about 40 μg/kg of odevixibat, or a pharmaceutically acceptable salt thereof, per dose of the pharmaceutical formulation.

57. The method of claim 54, wherein the subject is administered about 120 μg/kg of odevixibat, or a pharmaceutically acceptable salt thereof, per dose per dose of the pharmaceutical formulation.

58. The method of claim 54, wherein the subject is administered about 200 μg, about 400 μg, about 600 μg, or about 1200 μg of odevixibat, or a pharmaceutically acceptable salt thereof, per dose of the pharmaceutical formulation.


SEQUENCE LISTING (4 sequences)

SEQ ID NO: 1 - protein, 1251 aa, Homo sapiens
MSTERDSETTFDEDSQPNDEVVPYSDDETEDELDDQGSAVEPEQNRVNREAEENREPFRKECTWQVKANDRKYHEQPHFMNTKFLCIKESKYANNAIKTYKYNAFTFIPMNLFEQFKRAANLYFLALLILQAVPQISTLAWYTTLVPLLVVLGVTAIKDLVDDVARHKMDKEINNRTCEVIKDGRFKVAKWKEIQVGDVIRLKKNDFVPADILLLSSSEPNSLCYVETAELDGETNLKFKMSLEITDQYLQREDTLATFDGFIECEEPNNRLDKFTGTLFWRNTSFPLDADKILLRGCVIRNTDFCHGLVIFAGADTKIMKNSGKTRFKRTKIDYLMNYMVYTIFVVLILLSAGLAIGHAYWEAQVGNSSWYLYDGEDDTPSYRGFLIFWGYIIVLNTMVPISLYVSVEVIRLGQSHFINWDLQMYYAEKDTPAKARTTTLNEQLGQIHYIFSDKTGTLTQNIMTFKKCCINGQIYGDHRDASQHNHNKIEQVDFSWNTYADGKLAFYDHYLIEQIQSGKEPEVRQFFFLLAVCHTVMVDRTDGQLNYQAASPDEGALVNAARNFGFAFLARTQNTITISELGTERTYNVLAILDFNSDRKRMSIIVRTPEGNIKLYCKGADTVIYERLHRMNPTKQETQDALDIFANETLRTLCLCYKEIEEKEFTEWNKKFMAASVASTNRDEALDKVYEEIEKDLILLGATAIEDKLQDGVPETISKLAKADIKIWVLTGDKKETAENIGFACELLTEDTTICYGEDINSLLHARMENQRNRGGVYAKFAPPVQESFFPPGGNRALIITGSWLNEILLEKKTKRNKILKLKFPRTEEERRMRTQSKRRLEAKKEQRQKNFVDLACECSAVICCRVTPKQKAMVVDLVKRYKKAITLAIGDGANDVNMIKTAHIGVGISGQEGMQAVMSSDYSFAQFRYLQRLLLVHGRWSYIRMCKFLRYFFYKNFAFTLVHFWYSFFNGYSAQTAYEDWFITLYNVLYTSLPVLLMGLLDQDVSDKLSLRFPGLYIVGQRDLLFNYKRFFVSLLHGVLTSMILFFIPLGAYLQTVGQDGEAPSDYQSFAVTIASALVITVNFQIGLDTSYWTFVNAFSIFGSIALYFGIMFDFHSAGIHVLFPSAFQFTGTASNALRQPYIWLTIILAVAVCLLPVVAIRFLSMTIWPSESDKIQKHRKRLKAEEQWQRRQQVFRRGVSTRRSAYAFSHQRGYADLISSGRSIRKKRSPLDAIVADGTAEYRRTGDS

SEQ ID NO: 2 - DNA, 3756 bp, Homo sapiens
  source 1..3756: mol_type = genomic DNA
atgagtacagaaagagactcagaaacgacatttgacgaggattctcagcctaatgacgaagtggttccctacagtgatgatgaaacagaagatgaacttgatgaccaggggtctgctgttgaaccagaacaaaaccgagtcaacagggaagcagaggagaaccgggagccattcagaaaagaatgtacatggcaagtcaaagcaaacgatcgcaagtaccacgaacaacctcactttatgaacacaaaattcttgtgtattaaggagagtaaatatgcgaataatgcaattaaaacatacaagtacaacgcatttacctttataccaatgaatctgtttgagcagtttaagagagcagccaatttatatttcctggctcttcttatcttacaggcagttcctcaaatctctaccctggcttggtacaccacactagtgcccctgcttgtggtgctgggcgtcactgcaatcaaagacctggtggacgatgtggctcgccataaaatggataaggaaatcaacaataggacgtgtgaagtcattaaggatggcaggttcaaagttgctaagtggaaagaaattcaagttggagacgtcattcgtctgaaaaaaaatgattttgttccagctgacattctcctgctgtctagctctgagcctaacagcctctgctatgtggaaacagcagaactggatggagaaaccaatttaaaatttaagatgtcacttgaaatcacagaccagtacctccaaagagaagatacattggctacatttgatggttttattgaatgtgaagaacccaataacagactagataagtttacaggaacactattttggagaaacacaagttttcctttggatgctgataaaattttgttacgtggctgtgtaattaggaacaccgatttctgccacggcttagtcatttttgcaggtgctgacactaaaataatgaagaatagtgggaaaaccagatttaaaagaactaaaattgattacttgatgaactacatggtttacacgatctttgttgttcttattctgctttctgctggtcttgccatcggccatgcttattgggaagcacaggtgggcaattcctcttggtacctctatgatggagaagacgatacaccctcctaccgtggattcctcattttctggggctatatcattgttctcaacaccatggtacccatctctctctatgtcagcgtggaagtgattcgtcttggacagagtcacttcatcaactgggacctgcaaatgtactatgctgagaaggacacacccgcaaaagctagaaccaccacactcaatgaacagctcgggcagatccattatatcttctctgataagacggggacactcacacaaaatatcatgacctttaaaaagtgctgtatcaacgggcagatatatggggaccatcgggatgcctctcaacacaaccacaacaaaatagagcaagttgattttagctggaatacatatgctgatgggaagcttgcattttatgaccactatcttattgagcaaatccagtcagggaaagagccagaagtacgacagttcttcttcttgctcgcagtttgccacacagtcatggtggataggactgatggtcagctcaactaccaggcagcctctcccgatgaaggtgccctggtaaacgctgccaggaactttggctttgccttcctcgccaggacccagaacaccatcaccatcagtgaactgggcactgaaaggacttacaatgttcttgccattttggacttcaacagtgaccggaagcgaatgtctatcattgtaagaaccccagaaggcaatatcaagctttactgtaaaggtgctgacactgttatttatgaacggttacatcgaatgaatcctactaagcaagaaacacaggatgccctggatatctttgcaaatgaaactcttagaaccctatgcctttgctacaaggaaattgaagaaaaagaatttacagaatggaataaaaagtttatggctgccagtgtggcctccaccaaccgggacgaagctctggataaagtatatgaggagattgaaaaagacttaattctcctgggagctacagctattgaagacaagctacaggatggagttccagaaaccatttcaaaacttgcaaaagctgacattaagatctgggtgcttactggagacaaaaaggaaactgctgaaaatataggatttgcttgtgaacttctgactgaagacaccaccatctgctatggggaggatattaattctcttcttcatgcaaggatggaaaaccagaggaatagaggtggcgtctacgcaaagtttgcacctcctgtgcaggaatctttttttccacccggtggaaaccgtgccttaatcatcactggttcttggttgaatgaaattcttctcgagaaaaagaccaagagaaataagattctgaagctgaagttcccaagaacagaagaagaaagacggatgcggacccaaagtaaaaggaggctagaagctaagaaagagcagcggcagaaaaactttgtggacctggcctgcgagtgcagcgcagtcatctgctgccgcgtcacccccaagcagaaggccatggtggtggacctggtgaagaggtacaagaaagccatcacgctggccatcggagatggggccaatgacgtgaacatgatcaaaactgcccacattggcgttggaataagtggacaagaaggaatgcaagctgtcatgtcgagtgactattcctttgctcagttccgatatctgcagaggctactgctggtgcatggccgatggtcttacataaggatgtgcaagttcctacgatacttcttttacaaaaactttgcctttactttggttcatttctggtactccttcttcaatggctactctgcgcagactgcatacgaggattggttcatcaccctctacaacgtgctgtacaccagcctgcccgtgctcctcatggggctgctcgaccaggatgtgagtgacaaactgagcctccgattccctgggttatacatagtgggacaaagagacttactattcaactataagagattctttgtaagcttgttgcatggggtcctaacatcgatgatcctcttcttcatacctcttggagcttatctgcaaaccgtagggcaggatggagaggcaccttccgactaccagtcttttgccgtcaccattgcctctgctcttgtaataacagtcaatttccagattggcttggatacttcttattggacttttgtgaatgctttttcaatttttggaagcattgcactttattttggcatcatgtttgactttcatagtgctggaatacatgttctctttccatctgcatttcaatttacaggcacagcttcaaacgctctgagacagccatacatttggttaactatcatcctggctgttgctgtgtgcttactacccgtcgttgccattcgattcctgtcaatgaccatctggccatcagaaagtgataagatccagaagcatcgcaagcggttgaaggcggaggagcagtggcagcgacggcagcaggtgttccgccggggcgtgtcaacgcggcgctcggcctacgccttctcgcaccagcggggctacgcggacctcatctcctccgggcgcagcatccgcaagaagcgctcgccgcttgatgccatcgtggcggatggcaccgcggagtacaggcgcaccggggacagctga

SEQ ID NO: 3 - protein, 1321 aa, Homo sapiens
MSDSVILRSIKKFGEENDGFESDKSYNNDKKSRLQDEKKGDGVRVGFFQLFRFSSSTDIWLMFVGSLCAFLHGIAQPGVLLIFGTMTDVFIDYDVELQELQIPGKACVNNTIVWTNSSLNQNMTNGTRCGLLNIESEMIKFASYYAGIAVAVLITGYIQICFWVIAAARQIQKMRKFYFRRIMRMEIGWFDCNSVGELNTRFSDDINKINDAIADQMALFIQRMTSTICGFLLGFFRGWKLTLVIISVSPLIGIGAATIGLSVSKFTDYELKAYAKAGVVADEVISSMRTVAAFGGEKREVERYEKNLVFAQRWGIRKGIVMGFFTGFVWCLIFLCYALAFWYGSTLVLDEGEYTPGTLVQIFLSVIVGALNLGNASPCLEAFATGRAAATSIFETIDRKPIIDCMSEDGYKLDRIKGEIEFHNVTFHYPSRPEVKILNDLNMVIKPGEMTALVGPSGAGKSTALQLIQRFYDPCEGMVTVDGHDIRSLNIQWLRDQIGIVEQEPVLFSTTIAENIRYGREDATMEDIVQAAKEANAYNFIMDLPQQFDTLVGEGGGQMSGGQKQRVAIARALIRNPKILLLDMATSALDNESEAMVQEVLSKIQHGHTIISVAHRLSTVRAADTIIGFEHGTAVERGTHEELLERKGVYFTLVTLQSQGNQALNEEDIKDATEDDMLARTFSRGSYQDSLRASIRQRSKSQLSYLVHEPPLAVVDHKSTYEEDRKDKDIPVQEEVEPAPVRRILKFSAPEWPYMLVGSVGAAVNGTVTPLYAFLFSQILGTFSIPDKEEQRSQINGVCLLFVAMGCVSLFTQFLQGYAFAKSGELLTKRLRKFGFRAMLGQDIAWFDDLRNSPGALTTRLATDASQVQGAAGSQIGMIVNSFTNVTVAMIIAFSFSWKLSLVILCFFPFLALSGATQTRMLTGFASRDKQALEMVGQITNEALSNIRTVAGIGKERRFIEALETELEKPFKTAIQKANIYGFCFAFAQCIMFIANSASYRYGGYLISNEGLHFSYVFRVISAVVLSATALGRAFSYTPSYAKAKISAARFFQLLDRQPPISVYNTAGEKWDNFQGKIDFVDCKFTYPSRPDSQVLNGLSVSISPGQTLAFVGSSGCGKSTSIQLLERFYDPDQGKVMIDGHDSKKVNVQFLRSNIGIVSQEPVLFACSIMDNIKYGDNTKEIPMERVIAAAKQAQLHDFVMSLPEKYETNVGSQGSQLSRGEKQRIAIARAIVRDPKILLLDEATSALDTESEKTVQVALDKAREGRTCIVIAHRLSTIQNADIIAVMAQGVVIEKGTHEELMAQKGAYYKLVTTGSPIS

SEQ ID NO: 4 - DNA, 3966 bp, Homo sapiens
  source 1..3966: mol_type = genomic DNA
atgtctgactcagtaattcttcgaagtataaagaaatttggagaggagaatgatggttttgagtcagataaatcatataataatgataagaaatcaaggttacaagatgagaagaaaggtgatggcgttagagttggcttctttcaattgtttcggttttcttcatcaactgacatttggctgatgtttgtgggaagtttgtgtgcatttctccatggaatagcccagccaggcgtgctactcatttttggcacaatgacagatgtttttattgactacgacgttgagttacaagaactccagattccaggaaaagcatgtgtgaataacaccattgtatggactaacagttccctcaaccagaacatgacaaatggaacacgttgtgggttgctgaacatcgagagcgaaatgatcaaatttgccagttactatgctggaattgctgtcgcagtacttatcacaggatatattcaaatatgcttttgggtcattgccgcagctcgtcagatacagaaaatgagaaaattttactttaggagaataatgagaatggaaatagggtggtttgactgcaattcagtgggggagctgaatacaagattctctgatgatattaataaaatcaatgatgccatagctgaccaaatggcccttttcattcagcgcatgacctcgaccatctgtggtttcctgttgggatttttcaggggttggaaactgaccttggttattatttctgtcagccctctcattgggattggagcagccaccattggtctgagtgtgtccaagtttacggactatgagctgaaggcctatgccaaagcaggggtggtggctgatgaagtcatttcatcaatgagaacagtggctgcttttggtggtgagaaaagagaggttgaaaggtatgagaaaaatcttgtgttcgcccagcgttggggaattagaaaaggaatagtgatgggattctttactggattcgtgtggtgtctcatctttttgtgttatgcactggccttctggtacggctccacacttgtcctggatgaaggagaatatacaccaggaacccttgtccagattttcctcagtgtcatagtaggagctttaaatcttggcaatgcctctccttgtttggaagcctttgcaactggacgtgcagcagccaccagcatttttgagacaatagacaggaaacccatcattgactgcatgtcagaagatggttacaagttggatcgaatcaagggtgaaattgaattccataatgtgaccttccattatccttccagaccagaggtgaagattctaaatgacctcaacatggtcattaaaccaggggaaatgacagctctggtaggacccagtggagctggaaaaagtacagcactgcaactcattcagcgattctatgacccctgtgaaggaatggtgaccgtggatggccatgacattcgctctcttaacattcagtggcttagagatcagattgggatagtggagcaagagccagttctgttctctaccaccattgcagaaaatattcgctatggcagagaagatgcaacaatggaagacatagtccaagctgccaaggaggccaatgcctacaacttcatcatggacctgccacagcaatttgacacccttgttggagaaggaggaggccagatgagtggtggccagaaacaaagggtagctatcgccagagccctcatccgaaatcccaagattctgcttttggacatggccacctcagctctggacaatgagagtgaagccatggtgcaagaagtgctgagtaagattcagcatgggcacacaatcatttcagttgctcatcgcttgtctacggtcagagctgcagataccatcattggttttgaacatggcactgcagtggaaagagggacccatgaagaattactggaaaggaaaggtgtttacttcactctagtgactttgcaaagccagggaaatcaagctcttaatgaagaggacataaaggatgcaactgaagatgacatgcttgcgaggacctttagcagagggagctaccaggatagtttaagggcttccatccggcaacgctccaagtctcagctttcttacctggtgcacgaacctccattagctgttgtagatcataagtctacctatgaagaagatagaaaggacaaggacattcctgtgcaggaagaagttgaacctgccccagttaggaggattctgaaattcagtgctccagaatggccctacatgctggtagggtctgtgggtgcagctgtgaacgggacagtcacacccttgtatgcctttttattcagccagattcttgggactttttcaattcctgataaagaggaacaaaggtcacagatcaatggtgtgtgcctactttttgtagcaatgggctgtgtatctcttttcacccaatttctacagggatatgcctttgctaaatctggggagctcctaacaaaaaggctacgtaaatttggtttcagggcaatgctggggcaagatattgcctggtttgatgacctcagaaatagccctggagcattgacaacaagacttgctacagatgcttcccaagttcaaggggctgccggctctcagatcgggatgatagtcaattccttcactaacgtcactgtggccatgatcattgccttctcctttagctggaagctgagcctggtcatcttgtgcttcttccccttcttggctttatcaggagccacacagaccaggatgttgacaggatttgcctctcgagataagcaggccctggagatggtgggacagattacaaatgaagccctcagtaacatccgcactgttgctggaattggaaaggagaggcggttcattgaagcacttgagactgagctggagaagcccttcaagacagccattcagaaagccaatatttacggattctgctttgcctttgcccagtgcatcatgtttattgcgaattctgcttcctacagatatggaggttacttaatctccaatgaggggctccatttcagctatgtgttcagggtgatctctgcagttgtactgagtgcaacagctcttggaagagccttctcttacaccccaagttatgcaaaagctaaaatatcagctgcacgcttttttcaactgctggaccgacaacccccaatcagtgtatacaatactgcaggtgaaaaatgggacaacttccaggggaagattgattttgttgattgtaaatttacatatccttctcgacctgactcgcaagttctgaatggtctctcagtgtcgattagtccagggcagacactggcgtttgttgggagcagtggatgtggcaaaagcactagcattcagctgttggaacgtttctatgatcctgatcaagggaaggtgatgatagatggtcatgacagcaaaaaagtaaatgtccagttcctccgctcaaacattggaattgtttcccaggaaccagtgttgtttgcctgtagcataatggacaatatcaagtatggagacaacaccaaagaaattcccatggaaagagtcatagcagctgcaaaacaggctcagctgcatgattttgtcatgtcactcccagagaaatatgaaactaacgttgggtcccaggggtctcaactctctagaggggagaaacaacgcattgctattgctcgggccattgtacgagatcctaaaatcttgctactagatgaagccacttctgccttagacacagaaagtgaaaagacggtgcaggttgctctagacaaagccagagagggtcggacctgcattgtcattgcccatcgcttgtccaccatccagaacgcggatatcattgctgtcatggcacagggggtggtgattgaaaaggggacccatgaagaactgatggcccaaaaaggagcctactacaaactagtcaccactggatcccccatcagttga